(19) United States
(12) Patent Application Publication
(10) Pub. No.: US 2026/0337625 A1
(43) Pub. Date: Oct. 29, 2026

(54) IN-FRAME DELETIONS FOR MODULATING PROTEIN ACTIVITY IN PLANTS

(71) Applicant: Inari Agriculture Technology, Inc., Cambridge, MA (US)

(72) Inventors: Ross Everett ALTMAN, Boston, MA (US); Adam Patrick JOYCE, Stow, MA (US); Wenbin MEI, Lexington, MA (US); Jeremy David PARDO, Newton Highlands, MA (US)

(73) Assignee: Inari Agriculture Technology, Inc., Cambridge, MA (US)

(21) Appl. No.: 19/704,087

(22) Filed: Jun. 10, 2026

Related U.S. Application Data
(63) Continuation of application No. PCT/US2024/059654, filed on Dec. 11, 2024.
(60) Provisional application No. 63/609,266, filed on Dec. 12, 2023.

(51) Int. Cl. C12N 15/82; C12N 9/22; C12N 15/11

(52) U.S. Cl. CPC C12N 15/8251; C12N 9/226; C12N 15/111; C12N 15/8218; C12N 2310/20

(57) ABSTRACT

Provided herein are methods for modulating activity of a protein of interest and generating allelic variants of a plant gene using gene editing. In some embodiments, the provided methods comprise providing a gene editing system to a plant or part thereof, wherein editing by the gene editing system results in the deletion of one or more amino acids in the protein of interest. In some embodiments, the provided methods further comprise selecting progeny from an edited plant that exhibits an improvement in an agronomic trait due to the deletion of one or more amino acids in the protein of interest. The methods find use in accelerating breeding of new plant varieties with improved agronomic traits.

### CROSS-REFERENCE TO RELATED APPLICATIONS

This application is a continuation of International Application No. PCT/US2024/059654, filed internationally on Dec. 11, 2024, which claims priority from U.S. Provisional Application No. 63/609,266 filed Dec. 12, 2023, the contents of which are incorporated by reference in their entirety.

### FIELD

Aspects of this disclosure relate generally to biotechnology, in particular methods for modulating activity of a protein of interest and generating allelic variants of a plant gene using gene editing. In some aspects, the provided methods comprise providing a gene editing system to a plant or part thereof, wherein editing by the gene editing system results in the deletion of one or more amino acids in the protein of interest. In some aspects, the provided methods further comprise selecting progeny from an edited plant that exhibits an improvement in an agronomic trait due to the deletion of one or more amino acids in the protein of interest. The methods find use in accelerating breeding of new plant varieties with improved agronomic traits.

### BACKGROUND

There is a perpetual need for a stable food supply. While traditional breeding methods have been able to keep pace with demand so far, continued population growth, limited arable land and a changing climate indicate a fast-approaching need for increasing yield and productivity. Traditional breeding methods rely on the selection of trait variants that arise from random mutations and require multiple generations to produce an improved plant variety. Further, the limited natural variation in elite crops produces an additional breeding bottleneck due to a lack of functional diversity for trait selection.

The advent of gene editing technology, such as CRISPR/Cas systems, has enabled targeted modifications of plant genomes in a single generation. To date, the most common application of gene editing technology for crop improvement has been the generation of loss-of-function mutants. However, for many genes underlying agronomic traits, loss-of-function mutations can result in severe phenotypes which can pose additional challenges for breeding, such as homozygous lethality. There is an ongoing need for methods that can produce a spectrum of intermediate phenotypes for breeding new plant varieties with improved agronomic traits. Provided herein are embodiments that meet such needs.

### SUMMARY

An aspect of the disclosure includes a method of modulating the activity of a protein of interest in a plant or plant part comprising providing a Cas nuclease and a guide RNA for the Cas nuclease to the plant or plant part, wherein the guide RNA is complementary to a target sequence in a target site, wherein the target site comprises one or more codons of a plant gene encoding a protein of interest, wherein editing by the Cas nuclease and the guide RNA results in deletion of one or more amino acids in the protein of interest.

Another aspect of the disclosure includes a method of creating an allelic series of a plant gene comprising providing a Cas nuclease and a guide RNA for the Cas nuclease to each of a plurality of plants, wherein the guide RNA is complementary to a target sequence in a target site, wherein the target site comprises one or more codons of a plant gene encoding a protein of interest, wherein editing by the Cas nuclease and the guide RNA generates a series of different amino acid deletions in the protein of interest across the plurality of plants. An additional embodiment of this aspect includes selecting progeny of the plurality of plants, wherein each of the progeny contain different amino acid deletions in the protein of interest. A further embodiment of this aspect includes the selected progeny exhibiting a range of phenotypes. A further embodiment of this aspect includes the range of phenotypes being a spectrum of intermediate phenotypes for a quantitative agronomic trait. A further embodiment of this aspect includes one or more the selected progeny exhibiting an improvement in an agronomic trait.

In some of any embodiments, the method further comprises selecting a progeny from the plant, wherein the progeny exhibits an improvement in an agronomic trait due to the deletion of one or more amino acids in the protein of interest. In some of any embodiments, the method further comprises backcrossing the selected progeny to remove the Cas nuclease and the guide RNA. In some of any embodiments, the selected progeny no longer contains the Cas nuclease and the guide RNA.

In some of any embodiments, the method comprises providing two or more, three or more, four or more, five or more, six or more, seven or more, or eight or more guide RNAs to the plurality of plants. In some embodiments, each of the guide RNAs targets one or more target sites within a single exon of the plant gene encoding the protein of interest. In some embodiments, each of the guide RNAs targets one or more target sites in different exons of the plant gene encoding the protein of interest. In some of any embodiments, the guide RNA targets an evolutionary conserved sequence in the protein of interest.

In some of any embodiments, the deletion of one or more amino acids in the protein of interest comprises an in-frame deletion. In some embodiments, the in-frame deletion comprises a deletion of a multiple of 3 nucleotides that results in the deletion of one or more codons of the plant gene encoding the protein of interest. In some embodiments, the in-frame deletion further comprises a partial deletion of one codon and a synonymous mutation in an adjacent codon. In some embodiments, the in-frame deletion creates an allelic variant of the plant gene encoding the protein of interest.

In some embodiments, the in-frame deletion comprises a deletion of two, three, four, five, six, seven, eight, nine, or ten codons from the plant gene encoding the protein of interest. In some embodiments, the in-frame deletion comprises a deletion of one or more codons encoding cysteine from the plant gene encoding the protein of interest. In some embodiments, the in-frame deletion comprises a deletion of a stop codon from the plant gene encoding the protein of interest. In some embodiments, deletion of the stop codon results in an increase in the length of the protein of interest. In some embodiments, the in-frame deletion comprises a deletion of three, six, nine, 12, 15, 18, 21, 24, 27, or 30 nucleotides comprising one or more codons from the plant gene encoding the protein of interest.

In some of any embodiments, editing by the Cas nuclease and the guide RNA increases or reduces the biological activity of the protein of interest. In some of any embodiments, editing by the Cas nuclease and the guide RNA results in a different structural isoform of the protein of interest. In some of any embodiments, editing by the Cas nuclease and the guide RNA results in truncation of the protein of interest. In some of any embodiments, editing by the Cas nuclease and the guide RNA results in a N-terminal or C-terminal truncation of the protein of interest. In some of any embodiments, editing by the Cas nuclease and the guide RNA removes a site of a post-translational modification or co-translational modification from the protein of interest. In some of any embodiments, editing by the Cas nuclease and the guide RNA results in decreased post-translational modification or co-translational modification of the protein of interest.

In some of any embodiments, editing by the Cas nuclease and the guide RNA increases turnover of the protein of interest. In some of any embodiments, editing by the Cas nuclease and the guide RNA decreases turnover of the protein of interest. In some of any embodiments, editing by the Cas nuclease and the guide RNA stabilizes an mRNA transcript encoding the protein of interest. In some of any embodiments, editing by the Cas nuclease and the guide RNA destabilizes an mRNA transcript encoding the protein of interest. In some of any embodiments, editing by the Cas nuclease and the guide RNA results in alternative splicing of an mRNA transcript encoding the protein of interest. In some of any embodiments, editing by the Cas nuclease and the guide RNA removes a binding site on the protein of interest. In some embodiments, the binding site removed from the protein of interest is for a protein, nucleic acid, lipid, carbohydrate or small molecule. In some of any embodiments, editing by the Cas nuclease and the guide RNA alters the subcellular localization of the protein of interest. In some of any embodiments, editing by the Cas nuclease and the guide RNA alters the intercellular transport of the protein of interest.

In some of any embodiments, editing by the Cas nuclease and the guide RNA decreases the level of the protein of interest. In some embodiments, the level of the protein of interest is decreased by at least 1%, at least 5%, at least 10%, at least 15%, at least 20%, at least 30%, at least 40%, at least 50%, at least 70%, at least 80%, or at least 90%. In some of any embodiments, editing by the Cas nuclease and the guide RNA increases the level of the protein of interest. In some embodiments, the level of the protein of interest is increased by at least 1%, at least 5%, at least 10%, at least 15%, at least 20%, at least 30%, at least 40%, at least 50%, at least 70%, at least 80%, at least 90%, at least 100%, at least 200%, at least 300%, at least 400%, at least 500%, at least 600%, at least 700%, at least 800%, or up to 900%.

In some of any embodiments, the Cas nuclease is a Cas9, Cas12a (Cpf1), Cas12c, Cas12d, Cas12e, Cas12b, Cas12h, Cas12i, Cas12j, Cas12f, or an engineered Cas nuclease. In some of any embodiments, the Cas nuclease is a Cas12i nuclease. In some embodiments, the Cas12i nuclease is derived from Lachnospiraceae. In some of any embodiments, the Cas nuclease is a Cas12a (Cpf1) nuclease. In some embodiments, the Cas12a nuclease is derived from Francisella novicida, Acidaminococcus sp, or Lachnospiraceae.

In some of any embodiments, expression of the Cas nuclease is driven by a constitutive, inducible, tissue-specific or developmental promoter. In some of any embodiments, the Cas nuclease is codon-optimized for expression in dicots or monocots. In some of any embodiments, the Cas nuclease and the guide RNA are provided by particle bombardment of a plant, plant part or plurality of plants. In some of any embodiments, the Cas nuclease and the guide RNA are provided by Agrobacterium-mediated transformation of the plant, plant part or plurality of plants. In some of any embodiments, the guide RNA is provided by application of a composition comprising the guide RNA to the plant, plant part or plurality of plants.

In some of any embodiments, the method further comprises providing a donor template. In some embodiments, insertion of the donor template results in the precise deletion of one or more codons in the plant gene encoding the protein of interest. In some of any embodiments, the Cas nuclease is associated with a reverse transcriptase.

In some of any embodiments, the agronomic trait is selected from the group consisting of herbicide tolerance, disease resistance, insect or pest resistance, increased grain yield, increased oil, altered plant maturity, enhanced stress tolerance, and altered morphological characteristics. In some of any embodiments, the plant gene encoding the protein of interest is a developmental gene. In some of any embodiments, the plant gene encoding the protein of interest exhibits dosage sensitivity. In some of any embodiments, the plant gene encoding the protein of interest regulates the cell cycle, cell growth, stem cell fate or hormone signaling. In some of any embodiments, the protein of interest is a signaling peptide or transcription factor. In some of any embodiments, low or high levels of the protein of interest are associated with an agronomically undesirable phenotype. In some of any embodiments, the protein of interest is involved in drought tolerance, disease resistance, pest resistance, stress tolerance, yield, shape, odor, texture, metabolite production, pigmentation, seed fecundity, endoreduplication, sugar content, pH, improved shelf life or storability, cell differentiation, branching, plant height, time to fruit set, or light reception.

In some of any embodiments, the plant or plurality of plants is selected from the group consisting of maize, rice, sorghum, rye, barley, wheat, pearl millet, foxtail millet, proso millet, fonio millet, teff, flax, oats, sugarcane, turfgrass, switchgrass, soybean, canola, alfalfa, sunflower, cotton, tobacco, tomato, peanut, potato, cannabis, a forage crop, an industrial crop, a woody crop, a biomass crop, and Arabidopsis. In some of any embodiments, the plant part is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings. In some of any embodiments, the plant, plant part or plurality of plants is a crop plant. In some of any embodiments, the plant, plant part or plurality of plants is a monocot or a dicot.

Also provided herein is a plant produced by the method of some of any embodiments. Also provided herein is a progeny of the plant produced by the method of some of any embodiments. Also provided herein is a seed of the plant produced by the method of some of any embodiments or a seed of the progeny of the plant produced by the method of some of any embodiments.

### DETAILED DESCRIPTION

The application of gene editing for improving agronomic traits in crop plants has yet to realize its full potential. Gene editing in crops has largely focused on gene disruption to generate loss-of-function mutants for breeding. However, more sophisticated changes in gene dosage or gene product could provide more functional diversity and a wider phenotypic range for quantitative agronomic traits, such as yield and flowering time. These types of changes can be accomplished by deleting a multiple of three nucleotides that removes one or more amino acid codons within a protein coding sequence, herein referred to as an “in-frame deletion.” Rather than abrogate the activity of a protein of interest, in-frame deletions in a protein of interest can tune protein activity and provide a means to create intermediate phenotypes for optimizing key agronomic traits.

A variety of in-frame deletions in the same gene can also be created using gene editing to create an allelic series. As used herein, an “allelic series” refers to a range of phenotypes observable due to variable mutations across multiple alleles for a given gene. The range of phenotypes from an allelic series of a gene are typically intermediate in scope compared to the phenotypes produced by the wildtype gene and a loss-of-function mutation in the gene. Thus, using in-frame deletions to create an allelic series can increase the functional diversity of the edited gene and produce a range of intermediate phenotypes to accelerate the breeding of new plant varieties with improved agronomic traits.

Further, the ability to analyze multiple alleles for a gene is highly useful for understanding gene function, especially for genes underlying important agronomic traits in crop plants since they often produce complex traits or are lethal when homozygous for a loss-of-function mutation (Campbell et al. (2019) Nature Scientific Reports, 9, 14757). In particular, the predictability of gene editing outcomes in plant breeding would be substantially enhanced by the increased understanding of gene function gained from the creation and analysis of allelic series (Scheben and Edwards (2018) Current Opinion in Plant Biology, 45:218-225).

Moreover, the sequence specific targeting capabilities of gene editing systems, such as a CRISPR/Cas gene editing system, enables the introduction of in-frame deletions into an evolutionarily conserved sequence of a protein of interest. As used herein, the term “conserved sequence” refers to a nucleotide or amino acid sequence that is identical or similar across a variety of species or organisms. For example, orthologous proteins are often identified from conserved sequences revealed by multiple local sequence alignments spanning a variety of species. Sequence conservation typically indicates amino acids that are essential to the structure, stability, or activity of a protein. As a result, the introduction of in-frame deletions into a conserved sequence can also serve as a structure-function analysis for the protein of interest. Moreover, the targeting of an evolutionarily conserved sequence increases the likelihood that a particular mutation identified through this approach will have a similar effect in orthologous proteins containing the conserved sequence in other plant species.

Provided herein are methods for modulating the activity of a protein of interest using a gene editing system to delete one or more codons in a gene sequence encoding the protein of interest. In some aspects, the deletion of a multiple of three nucleotides that removes one more codons in a plant gene sequence encoding the protein of interest comprises an in-frame deletion. In some aspects, the in-frame deletion results in the deletion of one or more amino acids in the protein of interest. In some aspects, the in-frame deletion alters the structure of the protein of interest. In some aspects, the in-frame deletion alters the function of the protein of interest. In some aspects, the in-frame deletion is introduced into an evolutionarily conserved sequence in the protein of interest. In some aspects, the in-frame deletion creates an allelic variant of the protein of interest. In some aspects, the allelic variant of the protein of interest improves an agronomic trait.

Also provided herein are methods for creating an allelic series of a plant gene encoding a protein of interest using a gene editing system to generate a series of in-frame deletions in the plant gene across a plurality of plants. As used herein, the phrase “plurality of plants” refers to more than one plant that are genetically similar. In some aspects, the series of in-frame deletions results in a series of amino acid deletions in the protein of interest across the plurality of plants. In some aspects, the series of in-frame deletions results in variable functionality of the protein of interest across the plurality of plants. In some aspects, the series of in-frame deletions comprises an allelic series of a plant gene encoding a protein of interest. In some aspects, the plurality of plants comprising the allelic series exhibit a range of phenotypes. In some aspects, one or more of the plurality of plants comprising the allelic series exhibit an improvement in an agronomic trait.

Also provided are methods further comprising selecting progeny of edited plants, wherein the selected progeny exhibit an improvement in an agronomic trait due to an in-frame deletion in a plant gene and the corresponding deletion of one or more amino acids in the protein of interest. In some aspects, the provided methods further comprise selecting a plurality of progeny from the plurality of edited plants wherein the selected plurality of progeny exhibit a range of phenotypes comprising improvements in an agronomic trait. In some aspects, provided are edited plants, as well as progeny and seed derived from said plants, produced by any one of the methods provided herein. In some aspects, the provided methods and plants can be used to accelerate breeding of new plant varieties with improved agronomic traits.

The section headings used herein are for organizational purposes only and are not to be construed as limiting the subject matter described.

### I. METHODS OF INTRODUCING IN-FRAME DELETIONS FOR MODULATING PROTEIN ACTIVITY AND CREATING ALLELIC SERIES

Provided herein are methods for modulating activity of a protein of interest through deletion of a multiple of three nucleotides that removes one or more amino acid codons in a plant gene encoding the protein of interest, herein referred to as an “in-frame deletion.” In some aspects, the provided methods comprise providing a gene editing system to a plant or part thereof, wherein the gene editing system targets a target site in one or more codons of a plant gene encoding the protein of interest, wherein editing by the gene editing system results in the deletion of one or more amino acids in the protein of interest. In some aspects, the provided methods further comprise selecting progeny from an edited plant that exhibits an improvement in an agronomic trait due to the deletion of one or more amino acids in the protein of interest. Also provided are plants, as well as progeny and seed of said plants, produced by any of the methods disclosed herein.

Also provided herein are methods for creating an allelic series of a plant gene using gene editing to generate a variety of distinct in-frame deletions in the plant gene across a plurality of plants. In some aspects, the provided methods comprise providing each of a plurality of plants with a gene editing system, wherein the gene editing system targets a target site in one or more codons of a plant gene encoding a protein of interest, wherein editing by the gene editing system generates a series of different amino acid deletions in the protein of interest across the plurality of plants.

In some aspects, the provided methods further comprise selecting progeny of the plurality of edited plants that exhibit a range of phenotypes due to different amino acid deletions in the protein of interest. In some aspects, the provided methods further comprise selecting progeny from an edited plant that exhibits an improvement in an agronomic trait due to the deletion of one or more amino acids in the protein of interest. In some aspects, the provided methods produce a series of allelic variants comprising a plurality of plants with distinct in-frame deletions in the plant gene encoding the protein of interest. Also provided are plants, as well as progeny and seed of said plants, produced by any of the methods disclosed herein. In some aspects, the provided methods and plants generate a spectrum of variation in a plant gene for use in plant breeding and crop improvement.

### A. Proteins of Interest

As described herein, a protein of interest can be any protein encoded by a gene in a plant genome. Generally, a protein of interest underlies an important agronomic trait (e.g., increased yield, improved nitrogen use efficiency, flowering time). As used herein, the term “agronomic trait” refers to a trait deemed beneficial to crop plants, such as traits related to plant growth, crop maintenance, and the consumption or utilization of the crop plant. In addition to improving or maintaining the health of a crop germplasm, traits that improve the growth or maintenance of crop plants may provide the plant additional energy for producing the consumptive product of the specific crop. For example, improved branching may allow a fruiting crop better access to sunlight, which in turn allows more energy capture that supplies metabolic material for fruit production.

Bioinformatic tools can assist in the identification of putative proteins that may benefit plant agronomic traits in a modified state. There are extensive databases available for predicting or otherwise identifying these proteins, such as those built on microarray data, RNA-seq results, general genomic and transcriptomic functional annotation, and metabolomics. Various bioinformatic techniques continue to evolve that assist in this predictive work as well, such as improving genome assembly capabilities. A list of plant bioinformatic databases accessible through open access is available, for example, in Gomez-Casati et al. (2018) Curr. Issues Mol. Biol. 27: 89-104.

Proteins of interest can also be identified by the presence of evolutionarily conserved sequences or domains. Conserved sequences can be identified through any method known to one of ordinary skill in the art. For example, the nucleotide or protein sequence of a protein of interest can be aligned against nucleotide or protein sequence databases to identify regions of similarity using publicly available search tools (e.g., BLAST: https://blast.ncbi.nlm.nih.gov/Blast.cgi.) or bioinformatics tools that can access publicly available genomes (e.g., Python or R).

The National Center for Biotechnology information (NCBI) has also developed the Conserved Domain Database (CDD) dedicated to the identification of conserved protein sequence domains (see Marchler-Bauer A et al. (2015), “CDD: NCBI's conserved domain database,” Nucleic Acids Res. 43(D)222-6.) A user-friendly interface is available at https://www.ncbi.nlm.nih.gov/Structure/cdd/wrpsb.cgi. Based on the input sequence, local multiple sequence alignments that span a variety of organisms are used to identify a sequence region that contains the same, or similar, patterns of amino acids. The CDD assesses the extent of conservation using a position-specific scoring matrix (PSSM), which calculates the frequency of each of the various amino acids that are present in a given position of a multiple sequence alignment.

General categories of proteins of interest include, but are not limited to, those proteins involved in information, such as transcription factors including zinc finger-containing transcription factors, and those involved in communication, such as kinases and/or other signal-transduction factors. Proteins of interest generally include those affecting seed size, plant development, plant growth regulation, and yield improvement. Plant development and growth regulation also refer to the development and growth regulation of various parts of a plant, such as the flower, seed, root, leaf and shoot.

In some embodiments, the protein of interest is encoded by a plant gene that regulates plant growth and development. In some embodiments, the plant gene encoding the protein of interest is a developmental gene. In some embodiments the plant gene encoding the protein of interest exhibits dosage sensitivity (e.g., haploid insufficiency). In some embodiments, the plant gene encoding the protein of interest regulates the cell cycle. In some embodiments, the plant gene encoding the protein of interest regulates cell growth. In some embodiments, the plant gene encoding the protein of interest regulates stem cell fate. In some embodiments, the plant gene encoding the protein of interest regulates hormone signaling. In some embodiments, the protein of interest is a signaling peptide. In some embodiments, the protein of interest is a transcription factor. In some embodiments, the protein of interest is involved in drought tolerance, disease resistance, pest resistance, stress tolerance, yield, shape, odor, texture, metabolite production, pigmentation, seed fecundity, endoreduplication, sugar content, pH, improved shelf life or storability, cell differentiation, branching, plant height, time to fruit set, or light reception.

### B. In-Frame Deletions

As described herein, an in-frame deletion comprises a deletion of a multiple of three nucleotides that removes one or more amino acid codons in a plant gene encoding a protein of interest. In some embodiments, the in-frame deletion removes one or more amino acids in the translated protein corresponding to the plant gene of interest. In some embodiments, the in-frame deletion comprises the deletion of three or more nucleotides, comprising one or more codons, from the plant gene encoding the protein of interest. In some embodiments, the in-frame deletion comprises the deletion of three nucleotides from the plant gene encoding the protein of interest. In some embodiments, the three nucleotides that are deleted span more than one codon. For example, one nucleotide can be deleted from a first codon and two nucleotides can be deleted from a second adjacent codon to create an in-frame deletion. In some embodiments, the in-frame deletion comprises the deletion of six nucleotides from the plant gene encoding the protein of interest. In some embodiments, the in-frame deletion comprises the deletion of nine nucleotides from the plant gene encoding the protein of interest. In some embodiments, the in-frame deletion comprises the deletion of 12, 15, 18, 21, 24, 27, or 30 nucleotides from the plant gene encoding the protein of interest.

In some embodiments, the in-frame deletion comprises the deletion of one or more codons from the plant gene encoding the protein of interest. In some embodiments, the in-frame deletion comprises the deletion of one codon from the plant gene encoding the protein of interest. In some embodiments, the in-frame deletion comprises the deletion of two codons from the plant gene encoding the protein of interest. In some embodiments, the in-frame deletion comprises the deletion of three codons from the plant gene encoding the protein of interest. In some embodiments, the in-frame deletion comprises the deletion of four codons from the plant gene encoding the protein of interest. In some embodiments, the in-frame deletion comprises the deletion of five, six, seven, eight, nine or ten codons from the plant gene encoding the protein of interest. In some embodiments, the in-frame deletion comprises the deletion of one or more codons encoding cysteine from the plant gene encoding the protein of interest. In some embodiments, the in-frame deletion comprises the deletion of a stop codon from the plant gene encoding the protein of interest.

In some embodiments, the in-frame deletion further comprises a synonymous substitution of a proximal codon in the plant gene encoding the protein of interest (i.e., the codon sequence is modified, but the encoded amino acid remains the same). For example, the nucleotide sequence GGG CCC AAA codes for the amino acids Gly-Pro-Lys. The deletion of Pro can be accomplished by deleting the CCC codon resulting in the nucleotide sequence GGG AAA, which codes for Gly-Lys. Alternatively, the GCC sequence that spans the Gly and Pro codons (i.e., GG[G CC]C AAA) can be deleted resulting in the sequence GGC-AAA, which still codes for Gly-Lys. Since GGC also codes for Gly, the in-frame deletion resulted in a synonymous substitution for Gly and deletion of Pro. See Table 1 below for a codon table listing the codons that encode each amino acid.

TABLE 1
Codon Table

| | | Second Base in Codon | | | | | | | | | |
|---|---|---|---|---|---|---|---|---|---|---|---|
| | | T | | C | | A | | G | | | |
| First | T | TTT | Phe (F) | TCT | Ser (S) | TAT | Tyr (Y) | TGT | Cys (C) | T | Third |
| Base | | TTC | Phe (F) | TCC | Ser (S) | TAC | Tyr (Y) | TGC | Cys (C) | C | Base |
| in Codon | | TTA | Leu (L) | TCA | Ser (S) | TAA | STOP | TGA | STOP | A | in Codon |
| | | TTG | Leu (L) | TCG | Ser (S) | TAG | STOP | TGG | Trp (W) | G | |
| | C | CTT | Leu (L) | CCT | Pro (P) | CAT | His (H) | CGT | Arg (R) | T | |
| | | CTC | Leu (L) | CCC | Pro (P) | CAC | His (H) | CGC | Arg (R) | C | |
| | | CTA | Leu (L) | CCA | Pro (P) | CAA | Gln (Q) | CGA | Arg (R) | A | |
| | | CTG | Leu (L) | CCG | Pro (P) | CAG | Gln (Q) | CGG | Arg (R) | G | |
| | A | ATT | Ile (I) | ACT | Thr (T) | AAT | Asn (N) | AGT | Ser (S) | T | |
| | | ATC | Ile (I) | ACC | Thr (T) | AAC | Asn (N) | AGC | Ser (S) | C | |
| | | ATA | Ile (I) | ACA | Thr (T) | AAA | Lys (K) | AGA | Arg (R) | A | |
| | | ATG | Met (M) | ACG | Thr (T) | AAG | Lys (K) | AGG | Arg (R) | G | |
| | G | GTT | Val (V) | GCT | Ala (A) | GAT | Asp (D) | GGT | Gly (G) | T | |
| | | GTC | Val (V) | GCC | Ala (A) | GAC | Asp (D) | GGC | Gly (G) | C | |
| | | GTA | Val (V) | GCA | Ala (A) | GAA | Glu (E) | GGA | Gly (G) | A | |
| | | GTG | Val (V) | GCG | Ala (A) | GAG | Glu (E) | GGG | Gly (G) | G | |

In some embodiments, the in-frame deletion results in the deletion of one or more amino acids in the protein of interest. In some embodiments, the in-frame deletion comprises the deletion of one amino acid in the protein of interest. In some embodiments, the in-frame deletion comprises the deletion of two amino acids in the protein of interest. In some embodiments, the in-frame deletion comprises the deletion of three amino acids in the protein of interest. In some embodiments, the in-frame deletion comprises the deletion of four amino acids in the protein of interest. In some embodiments, the in-frame deletion comprises the deletion of five, six, seven, eight, nine or ten amino acids in the protein of interest. In some embodiments, the in-frame deletion comprises the deletion of one or more cysteines in the protein of interest.

In some embodiments, the in-frame deletion is located within a single exon of a plant gene encoding the protein of interest. In some embodiments, the in-frame deletion is located near the 5′ end of the exon of the plant gene encoding the protein of interest. In some embodiments, the in-frame deletion is located near the 3′ end of the exon of the plant gene encoding the protein of interest. In some embodiments, the in-frame deletion is in the middle of an exon of the plant gene encoding the protein of interest. In some embodiments, the in-frame deletion spans a splice junction connecting two exons of the plant gene encoding the protein of interest.

In some embodiments, the in-frame deletion results in the deletion of one or more amino acids anywhere along the length of the protein of interest. In some embodiments, one or more amino acids are deleted from the N-terminus of the protein of interest. In some embodiments, one or more amino acids are deleted from the C-terminus of the protein of interest. In some embodiments, one or more amino acids are deleted from the middle of the protein of interest. In some embodiments, the in-frame deletion is located within an evolutionarily conserved sequence region of the protein of interest. In some embodiments, the in-frame deletion in the evolutionarily conserved sequence region of the protein of interest alters the structure, stability, or activity of the protein of interest.

In some embodiments, the in-frame deletion alters the structure of the protein of interest. In some embodiments, the in-frame deletion results in alternative splicing of an mRNA encoding the protein of interest. In some embodiments, the in-frame deletion results in a different structural isoform of the protein of interest (e.g., a protein variant with a similar but not identical amino acid sequence). In some embodiments, the in-frame deletion results in a truncation of the protein of interest. In some embodiments, the in-frame deletion results in a N-terminal truncation of the protein of interest. In some embodiments, the in-frame deletion results in a C-terminal truncation of the protein of interest. In some embodiments, the in-frame deletion results in an increase in the length of the protein of interest. For example, by removing a stop codon in the gene encoding the protein of interest. In some embodiments, the in-frame deletion comprises the deletion of a stop codon in the gene encoding the protein of interest that results in an increase in the length of the protein of interest.

In some embodiments, the in-frame deletion reduces the biological activity of the protein of interest. In some embodiments, the in-frame deletion increases the biological activity of the protein of interest. For example, by removing the binding site for an inhibitory molecule. In some embodiments, the in-frame deletion removes a binding site (e.g., a binding site for a protein, nucleic acid, lipid, carbohydrate, or small molecule) from the protein of interest. In some embodiments, the in-frame deletion removes a site of protein-protein interaction from the protein of interest. In some embodiments, the in-frame deletion reduces the binding of the protein of interest to another protein. In some embodiments, the in-frame deletion removes a site of protein-DNA interaction from the protein of interest. In some embodiments, the in-frame deletion reduces the binding the protein of interest to DNA. In some embodiments, the in-frame deletion removes a site of post-translational modification from the protein of interest. In some embodiments, the in-frame deletion results in decreased post-translational modification of the protein of interest. In some embodiments, the in-frame deletion removes a site of co-translational modification from the protein of interest. In some embodiments, the in-frame deletion decreases the co-translational modification of the protein of interest.

In some embodiments, high levels of the protein of interest are associated with an agronomically undesirable phenotype. In some embodiments, the in-frame deletion decreases the level of the protein of interest. In some embodiments, the in-frame deletion destabilizes an mRNA transcript encoding the protein of interest. In some embodiments, the in-frame deletion increases turnover of the protein of interest. In some embodiments, the in-frame deletion decreases the level of the protein of interest by at least 1%, at least 5%, at least 10%, at least 15%, at least 20%, at least 30%, at least 40%, at least 50%, at least 70%, at least 80%, or at least 90%.

In some embodiments, low levels of the protein of interest are associated with an agronomically undesirable phenotype. In some embodiments, the in-frame deletion increases the level of the protein of interest. In some embodiments, the in-frame deletion stabilizes an mRNA transcript encoding the protein of interest. In some embodiments, the in-frame deletion decreases turnover of the protein of interest. In some embodiments, the in-frame deletion increases the level of the protein of interest by at least 1%, at least 5%, at least 10%, at least 15%, at least 20%, at least 30%, at least 40%, at least 50%, at least 70%, at least 80%, at least 90%, at least 100%, at least 200%, at least 300%, at least 400%, at least 500%, at least 600%, at least 700%, at least 800%, or up to 900%.

In some embodiments, the in-frame deletion alters the subcellular localization of the protein of interest. For example, the deletion of a nuclear localization signal (NLS) would reduce the nuclear localization of the protein of interest. In some embodiments, the in-frame deletion alters the intercellular transport of the protein of interest (e.g., disrupts membrane trafficking of a protein to different organelles).

### C. Allelic Series

As described herein, an “allelic series” refers to a range of phenotypes observable due to variable mutations across multiple alleles for a given gene. In some embodiments, editing by the provided gene editing system results in a series of different in-frame deletions in one or more codons of a plant gene encoding a protein of interest across a plurality of edited plants. In some embodiments, each of the in-frame deletions in a series result in the deletion of one or more different amino acids in the protein of interest. In some embodiments, the different in-frame deletions in a series occur in evolutionarily conserved sequences in the protein of interest.

In some embodiments, each of the different in-frame deletions in a series alters the functionality of the protein of interest to a different extent. In some embodiments, the different in-frame deletions in a series progressively decrease the functionality of the protein of interest. In some embodiments, the progressive decrease in the functionality of the protein of interest progressively increases the severity of a phenotype. In some embodiments, the different in-frame deletions result in a range of phenotypes. In some embodiments, the different in-frame deletions comprise an allelic series.

In some embodiments, the allelic series is for a plant gene underlying an agronomic trait. In some embodiments, the allelic series produces a series of variable phenotypes for an agronomic trait. In some embodiments, the allelic series produces a spectrum of intermediate phenotypes for a quantitative agronomic trait. For example, the allelic series could produce a plurality of edited plants that exhibit a range of heights, branching angles or times to flowering. The ability to tune these traits would enable the optimization of plant growth and yield for specific environments. In some embodiments, the allelic series increases the functional genetic diversity of an agronomic trait. In some embodiments, the allelic series contains one or more alleles that improve an agronomic trait. In some embodiments, the allelic series accelerates the breeding of improved plant varieties.

### D. CRISPR/Cas Gene Editing

Provided herein are methods of generating in-frame deletions in plants using gene editing systems comprising nucleases that can be targeted to specific sequences in a genome. The genetic editing of plants to improve agronomic traits may be accomplished through any method known to one skilled in the art, including methods comprising homing endonucleases, meganucleases, zinc finger nucleases, or transcription activator-like effector nucleases (TALENs), which require de novo protein engineering for every new target locus. However, in the last decade the use of CRISPR/Cas gene editing systems, comprising a Cas nuclease and a cognate guide RNA, has exploded in popularity due to the relative ease of engineering a guide RNA for sequence-specific nuclease targeting. In addition, the ever-expanding number of CRISPR/Cas variants that are available provide additional flexibility and specificity for targeted gene editing.

Accordingly, in some embodiments, the gene editing system provided to a plant, plant part or plurality of plants for introducing an in-frame deletion in a plant gene encoding a protein of interest is one of any described herein, for example, in Section II. In some embodiments, the gene editing system provided to the plant, plant part or plurality of plants is a CRISPR/Cas gene editing system comprising a Cas nuclease and cognate guide RNA. In some embodiments, the CRISPR/Cas system provided is one of any described herein, for example, in Section II-A.

In some embodiments, a Cas nuclease and a guide RNA for the Cas are provided to a plant or plant part, wherein the guide RNA is complementary to a target sequence in a target site, wherein the target site comprises one or more codons of a plant gene encoding a protein of interest, wherein editing by the Cas nuclease and the guide RNA results in the deletion of one or more amino acids in the protein of interest. In some embodiments, the target site comprises an evolutionary conserved sequence in the protein of interest.

In some embodiments, a Cas nuclease and a guide RNA for the Cas are provided to each of a plurality of plants, wherein the guide RNA is complementary to a target sequence in a target site, wherein the target site comprises one or more codons of a plant gene encoding a protein of interest, wherein editing by the Cas nuclease and the guide RNA generates a series of different amino acid deletions in the protein of interest across the plurality of plants. In some embodiments, the target site comprises an evolutionary conserved sequence in the protein of interest.

In some embodiments, multiple guide RNAs are provided to a plant or plant part. For example, multiple guide RNAs can be provided to a plant or plant part to increase the likelihood of achieving a desired edit due to the random nature of the DNA repair following breakage by the Cas nuclease. In some embodiments, two to eight guide RNAs are provided to the plant or plant part. In some embodiments, two or more, three or more, four or more, five or more, six or more, seven or more, or eight or more guide RNAs are provided to the plant or plant part. In some embodiments, each of the guide RNAs provided to the plant or plant part targets one or more target sites within a single exon of the plant gene encoding the protein of interest. In some embodiments, each of the guide RNAs provided to the plant or plant part targets one or more target sites in different exons of the plant gene encoding the protein of interest. In some embodiments, each of the guide RNAs provided to the plant or plant part targets one or more target sites in different plant genes encoding different proteins of interest.

In some embodiments, multiple guide RNAs are provided to each of a plurality of plants. For example, multiple guide RNAs can be provided to each of a plurality of plants to increase the range of variable mutations that are introduced following DNA cleavage by the Cas nuclease across the plurality of plants and thus increase the likelihood of creating an allelic series. In some embodiments, two to eight guide RNAs are provided to each of the plurality of plants. In some embodiments, two or more, three or more, four or more, five or more, six or more, seven or more, or eight or more guide RNAs are provided to each of the plurality of plants. In some embodiments, each of the guide RNAs targets provided to each of the plurality of plants targets one or more target sites within a single exon of the plant gene encoding the protein of interest. In some embodiments, each of the guide RNAs provided to each of the plurality of plants targets one or more target sites in different exons of the plant gene encoding the protein of interest. In some embodiments, each of the guide RNAs provided to each of the plurality of plants targets one or more target sites in different plant genes encoding different proteins of interest.

In some embodiments, the provided Cas nuclease is a Cas9, Cas12a (Cpf1), Cas12c, Cas12d, Cas12e, Cas12b, Cas12h, Cas12i, Cas12j, Cas12f, or an engineered Cas nuclease. In some embodiments, the provided Cas nuclease is a Cas12i nuclease. In some embodiments, the Cas12i nuclease is derived from Lachnospiraceae. In some embodiments, the Cas nuclease is a Cas12a (Cpf1 nuclease). In some embodiments, the Cas12a nuclease is derived from Francisella novicida, Acidaminococcus sp, or Lachnospiraceae. In some embodiments, the Cas12a nuclease is provided with an engineered Cas12a crRNA and a Cas12a tracrRNA.

While a guide RNA in a CRISPR/Cas system can be engineered to direct a Cas nuclease to generate a double-stranded break (DSB) in a specific DNA sequence, the DNA repair that follows is usually mediated through non-homologous end-joining (NHEJ). As a result, random insertions or deletions of various lengths (indels) are created at the break site. If a specific edit is desired (e.g., an in-frame deletion), sequencing of the randomly repaired target site is required to identify the desired edit. The additional provision of a donor template sequence encoding the desired edit, however, can promote homology directed repair (HDR) and result in precise edits to the target sequence.

Accordingly, in some embodiments, editing by the Cas nuclease and guide RNA results in precise in-frame deletions. In some embodiments, the provided CRISPR/Cas system further comprises a donor template. In some embodiments, the donor template enables Homology Directed Repair (HDR), as generally described in Section II-A. In some embodiments, the donor template is inserted at the target site. In some embodiments, the insertion of the donor template results in the precise alteration or deletion of one or more specific codons in the plant gene encoding the protein of interest. In some embodiments, the Cas nuclease is associated with a reverse transcriptase. In some embodiments, the associated reverse transcriptase enables prime editing, as generally described in Section II-A. In some embodiments, prime editing by the Cas nuclease and reverse transcriptase results in the precise alteration or deletion of one or more specific codons in the plant gene encoding the protein of interest.

### 1. Plant Expression Vectors

In some embodiments, one or more expression vectors comprising the Cas nuclease and guide RNA are used to provide the CRISPR/Cas system to the plant, plant part or plurality of plants. In some embodiments, the one or more expression vectors also comprise DNA elements that facilitate transformation of the plant, plant part or plurality of plants (e.g., T-DNA left and right borders). In some embodiments, the Cas nuclease is codon-optimized for expression in dicots or monocots.

In some embodiments, an expression vector includes a regulatory element such as a promoter. In some embodiments, the promoter is operably linked to one or more polynucleotides encoding the Cas nuclease or the guide RNA. In certain embodiments, the promoter is operably linked to a nucleotide sequence encoding multiple guide RNAs, wherein sequences encoding each guide RNA are separated by a cleavage site such as a nucleotide sequence encoding a microRNA recognition/cleavage site or a self-cleaving ribozyme (see, e.g., Ferré-D'Amare and Scott (2010) Cold Spring Harbor Perspectives Biol., October; 2(10):a003574). In some embodiments, the promoter is an RNA polymerase III promoter operably linked to a nucleotide sequence encoding one or more guide RNAs.

In some embodiments, expression of the Cas nuclease is driven by a constitutive, inducible, tissue-specific or developmental promoter. In certain embodiments, the promoter operably linked to one or more polynucleotides encoding the Cas nuclease is a constitutive promoter that drives gene expression in plant cells. Examples of constitutive promoters for use in plants include a Cauliflower Mosaic Virus (CaMV) 35S promoter as disclosed in U.S. Pat. Nos. 5,858,742 and 5,322,938, a rice actin promoter as disclosed in U.S. Pat. No. 5,641,876, a maize chloroplast aldolase promoter as disclosed in U.S. Pat. No. 7,151,204, and the nopaline synthase (NOS) and octopine synthase (OCS) promoters from Agrobacterium tumefaciens. Other contemplated promoters include cell-specific or tissue-specific or developmentally regulated promoters, for example, a promoter that limits the expression of the CRISPR/Cas system to germline or reproductive cells (e.g., promoters of genes encoding DNA ligases, recombinases, replicases, or other genes specifically expressed in germline or reproductive cells).

In some embodiments, an expression vector includes a selectable marker gene to allow convenient screening or selection of transformed cells or plants expressing the vector. Any suitable selectable marker gene known to one of ordinary skill in the art and/or described herein, for example in Section II-B-1, can be used. For example, an expression vector can include a polynucleotide encoding an antibiotic resistance gene such as the neomycin phosphotransferase II (nptII) gene, which confers resistance to kanamycin, or a polynucleotide encoding a detectable marker such as green fluorescent protein (GFP). In some embodiments, the polynucleotide encoding a selectable marker gene is operably linked to a promoter. In certain embodiments, expression of the selectable marker is driven by a constitutive or tissue-specific promoter. For example, a red fluorescent protein (RFP) can be operably linked to a seed-specific promoter which enables rapid, non-destructive screening for transformed plants (Shimada et al. Plant J. 2010 Feb. 1; 61(3):519-28).

### 2. Plant Transformation

In some embodiments, the Cas nuclease and guide RNA are provided using any one of the standard methods of plant transformation known to one of ordinary skill in the art and/or described herein, for example, in Section III. In some embodiments the Cas nuclease and guide RNA are provided by particle bombardment of a plant, plant part or plurality of plants. In some embodiments, the Cas nuclease and guide RNA are provided by Agrobacterium-mediated transformation of the plant, plant part or plurality of plants. In some embodiments, the Cas nuclease and guide RNA are provided as a single transgenic construct to the plant, plant part or plurality of plants. In some embodiments, the Cas nuclease and guide RNA are stably integrated into the genome of the plant, plant part or plurality of plants.

In certain embodiments, the CRISPR/Cas system is provided directly to the plant, plant part or plurality of plants as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, the guide RNA is provided by application of a composition comprising the guide RNA to the plant, plant part or plurality of plants. In certain embodiments, the plant, plant part or plurality of plants are transformed with a transgene that expresses a Cas nuclease (e.g., a Cas9 or Cas12 type nuclease). In certain embodiments, one or more Cas nucleases with unique PAM recognition sites can be used.

### 3. Progeny Selection

In some embodiments, progeny of the transformed plant or plurality of plants, or plants regenerated from a transformed plant part, are selected for further propagation and/or breeding. Plant selection and propagation can be accomplished using any one of the standard methods of plant selection and propagation known to one of ordinary skill in the art and/or described herein, for example, in Section IV.

Following plant transformation, expression of a selectable marker gene (e.g., green fluorescent protein or herbicide resistance) allows for preferential selection of transformed cells, tissues, and/or plants, using regeneration and selection methods well known in the art. Transformed plants expressing the selection marker (e.g., are fluorescent or can grow in the presence of a certain herbicide) are selected for further screening.

In practice, multiple independent transformation events (i.e., T1 plants) are identified and selected due to the random nature of T-DNA insertion into a plant genome (e.g., genomic location of insertion and insertion copy number) and intrinsic variability in editing efficacy (e.g., limitations of the gene editing system itself or designed elements such as gRNA sequences). Further, the random nature of DNA repair through non-homologous end joining (NHEJ), which is a feature of most gene editing systems, results in various mutations at the target site across the plurality of edited plants. Sequencing of the target site for each independent T1 plant line is therefore employed to identify the presence of a desired edit (e.g., an in-frame deletion). If a gene editing system is used that includes a donor template comprising the desired edit, such as HDR or prime editing, the presence of the desired edit is still confirmed by sequencing, but the amount of screening required is significantly reduced.

At the T1 stage, the transformation event is hemizygous and additional generations of the edited plants are needed to generate plants that are homozygous for the desired edit. The edited plants can also be backcrossed to an unedited parent plant or to an elite line to segregate away the CRISPR/Cas transgenic construct and generate transgene free plants comprising the desired gene edit.

Accordingly, in some embodiments, each of the selected progeny comprise a different in-frame deletion in the plant gene encoding the protein of interest. In some embodiments, each of the selected progeny comprise a different amino acid deletion in the protein of interest. In some embodiments, the selected progeny exhibits improvement in an agronomic trait due to deletion of one or more amino acids in the protein of interest. In some embodiments, the selected progeny exhibit a range of phenotypes. In some embodiments, the selected progeny comprise an allelic series. In some embodiments, the selected progeny no longer contain the Cas nuclease and the guide RNA(s). In some embodiments, a transgenic construct encoding the Cas nuclease and the guide RNA(s) is segregated away in following generations of the edited plants.

### E. Agronomic Trait Variants

In some embodiments, an in-frame deletion in the protein of interest results in the progeny of the edited plant exhibiting an intermediate phenotype in an agronomic trait compared to the phenotype of an unedited plant (i.e., contains the wildtype gene sequence for the protein of interest) and the phenotype of a plant with a null allele for the protein of interest (i.e., contains a mutation that renders the protein of interest non-functional). In some embodiments, the protein of interest regulates an agronomic trait such as drought tolerance, disease resistance, pest resistance, stress tolerance, yield, shape, odor, texture, metabolite production, nutrient absorption, pigmentation, seed fecundity, endoreduplication, sugar content, pH, improved shelf life or storability, cell differentiation, branching, plant height, time to fruit set, or light reception

In some embodiments, edited plants produced with the methods of the present disclosure exhibit improvement in one or more agronomic traits. In some embodiments, the edited plants are more resilient to damage. In some embodiments, the edited plants are more stable in the face of environmental change. In some embodiments, the edited plants help stabilize critical food webs. In some embodiments, the edited plants offer better nutritional value for human consumption or animal feed products. In some embodiments, the edited plants help address food security concerns.

In some embodiments, the plant, plant part or plurality of plants provided with a gene editing system (e.g., a CRISPR/Cas system) is a crop plant. In some embodiments, the plant, plant part or plurality of plants provided with a gene editing system is a monocot. In some embodiments, the plant, plant part or plurality of plants provided with a gene editing system is a dicot. In some embodiments, the plant or plurality of plants is selected from the group consisting of maize, rice, sorghum, rye, barley, wheat, sorghum, pearl millet, foxtail millet, proso millet, fonio millet, teff, oats, sugarcane, turfgrass, and switchgrass. In some embodiments, the plant or plurality of plants is selected from the group consisting of flax, canola, soybean, alfalfa, sunflower, cotton, tobacco, peanut, potato, cannabis, tomato, and Arabidopsis. In some embodiments, the plant or plurality of plants is selected from the group consisting of a forage crop, an industrial crop, a woody crop, and a biomass crop. In some embodiments, the plant part provided with a gene editing system is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

### II. GENE EDITING SYSTEMS

Provided herein are methods that employ gene editing systems comprising nucleases capable of introducing a double-strand break (“DSB”) in double-stranded DNA, such as in a target gene located within genomic DNA. Non-limiting examples of such gene editing systems include a CRISPR/Cas system comprising a Cas nuclease and cognate guide RNA (gRNA), a zinc-finger nuclease (ZFN), a transcription activator-like effector nuclease (TAL-effector nuclease), and a meganuclease. Additional nuclease variants that induce a single-strand break (“SSB”) in double-stranded DNA, such as nickases, have also been adopted for gene editing.

Exemplary components and features of the gene editing systems are provided below in the following subsections.

### A. CRISPR/Cas

CRISPR (Clustered Regularly Interspaced Short Palin-dromic Repeats)/Cas (CRISPR-associated) systems, or CRISPR systems, are adaptive defense systems originally discovered in bacteria and archaea. CRISPR systems use RNA-guided nucleases termed CRISPR-associated or “Cas” endonucleases (e.g., Cas9 or Cas12a (“Cpf1”)) to cleave foreign DNA. In a typical CRISPR/Cas system, a Cas endonuclease is directed to a target nucleotide sequence (e.g., a site in the genome that is to be sequence-edited) by sequence-specific, non-coding “guide RNAs” that target single- or double-stranded DNA sequences.

Two classes (Class 1 and Class 2) of CRISPR systems have been identified across a wide range of bacterial species. The well characterized Class 2 CRISPR systems use a single Cas endonuclease (rather than multiple Cas proteins). A Class 2 CRISPR system includes a Type II Cas endonuclease, such as Cas9, a CRISPR RNA (“crRNA”), and a trans-activating crRNA (“tracrRNA”). A “trans-activating crRNA” or “tracrRNA” is a trans-encoded small RNA that is partially homologous to repeats within a CRISPR array. The Cas9 crRNA contains a “spacer sequence”, typically an RNA sequence of about 20 nucleotides (in various embodiments this is 20, 21, 22, 23, 24, 25, or up to about 30 contiguous nucleotides in length) that corresponds to (e.g., is identical or nearly identical to, or alternatively is complementary or nearly complementary to) a target DNA sequence of about equivalent length. The Cas9 crRNA also contains a region that binds to the Cas9 tracrRNA to form a partially double-stranded structure which is cleaved by RNase III, resulting in a crRNA:tracrRNA hybrid or duplex. The crRNA: tracrRNA hybrid then directs the Cas9 endonuclease to recognize and cleave the target DNA sequence. In some CRISPR/Cas systems, a tracrRNA and crRNA (e.g., a crRNA including a spacer sequence) can both be included in a single chimeric nucleic acid referred to as a “single guide RNA” (sgRNA).

While CRISPR/Cas9 systems require both a tracrRNA and a crRNA for processing of the CRISPR array and nuclease-mediated cleavage of the target DNA sequence, CRISPR/Cas12a systems have been reported to function without a tracrRNA; see Zetsche et al. (2015) Cell, 163: 759-771 and U.S. Pat. No. 9,790,490. In other words, the naturally occurring CRISPR/Cas12a system was reported to require only the Cas12a (also known as “Cpf1”) nuclease and a Cas12a crRNA to cleave a target DNA sequence. Examples of Cas12a nucleases include AsCas12a or “AsCpf1” (from Acidaminococcus sp.) and LbCas12a or “LbCpf1” (from Lachnospiraceae bacteria).

Cas12a nucleases are Type V Cas endonucleases characterized as having only a RuvC nuclease domain, in contrast to Cas9 nucleases which have both RuvC and HNH nuclease domains. As a result, Cas12a nucleases are generally smaller proteins than Cas9 nucleases and can function with a smaller guide RNA (e.g., a crRNA having at least one spacer flanked by direct repeats); which are practical advantages in that the nuclease and guide RNAs are more economical to produce and potentially more easily delivered to a cell.

The genomic DNA sequence targeted for editing or modification must generally be adjacent to a “protospacer adjacent motif (“PAM”) that is specific for a given Cas endonuclease; however, PAM sequences are short and relatively non-specific, appearing throughout a given genome. Various methods (including in silico and/or wet lab methods) for identification of the appropriate PAM sequence are known in the art and are routine, and any convenient method can be used. For example, a PAM sequence can be identified using a PAM depletion assay.

In certain embodiments, one or more Cas endonucleases with unique PAM recognition sites can be used. CRISPR endonucleases identified from various prokaryotic species have unique PAM sequence requirements. Non-limiting examples of PAM sequences include 5′-NGG-3′ (Streptococcus pyogenes), 5′-NNAGAA-3′ (Streptococcus thermophilus, CRISPR1), 5′-NGGNG-3′ (Streptococcus thermophilus, CRISPR3), 5′-NNGRRT-3′ or 5′-NNGRR-3′ (Staphylococcus aureus, Cas9, SaCas9), and 5′-NNNGATT-3′ (Neisseria meningitidis). Examples of Cas12a PAM sequences include 5′-TTTN-3′, where N can be A, C, or G for the naturally occurring Acidaminococcus sp. BV3L6 Cpf1 (AsCpf1) and Lachnospiraceae bacterium ND2006 Cpf1 (LbCpf1) nucleases. Cas12a nucleases can also exhibit relaxed PAM recognition resulting in additional potential PAM sequences, including CTA, TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide).

In certain embodiments, an RNA-guided endonuclease that leaves a blunt end following cleavage of the target site is used. Blunt-end cutting RNA-guided endonucleases include Cas9, Cas12c, and Cas 12h (Yan et al., 2019). For example, Cas9 nucleases are associated with G-rich PAM sites (e.g., 5′-NGG-3′), and perform blunt-end cleaving of the target DNA at a location three nucleotides upstream (5′) from the PAM site.

In certain embodiments, an RNA-guided endonuclease that leaves a staggered single stranded DNA overhanging end following cleavage of the target site following cleavage of the target site is used. Staggered-end cutting RNA-guided endonucleases include Cas12a, Cas12b, and Cas12e. Specifically, CRISPR/12a (Cpf1) systems cleave the target DNA adjacent to a short T-rich PAM sequence (e.g., 5′-TTTN-3′) and introduce an offset or staggered double-strand break with a 5′ overhang that is four or five nucleotides long. For example, cleavage of a target DNA results in a five-nucleotide offset, or staggered cut, located 18 nucleotides downstream (3′) of the PAM site on the coding strand and 23 nucleotides downstream from the PAM site on the complimentary strand. The five-nucleotide overhang that results from such offset cleavage allows more precise genome editing by facilitating DNA insertion by homologous recombination as opposed to insertion at blunt-end cleaved DNA

For the purposes of gene editing, CRISPR arrays can be designed to contain one or multiple guide RNAs designed to target a DNA sequence for editing, where the guide RNA includes at least one spacer sequence that corresponds to a specific locus of about equivalent length in the target DNA; see, for example, Cong et al. (2013) Science, 339:819-823; and Ran et al. (2013) Nature Protocols, 8:2281-2308. As used herein “guide RNA” or “gRNA” refers to a nucleic acid that comprises a nucleotide sequence (sometimes referred to a “spacer sequence”) that corresponds to (e.g., is identical or nearly identical to, or alternatively is complementary or nearly complementary to) a target DNA sequence (e.g., a contiguous nucleotide sequence that is to be modified) in a genome. The guide RNA functions in part to direct the CRISPR nuclease to a specific genomic location.

In some embodiments, a gRNA is a CRISPR RNA (“crRNA”). For nucleases (such as a Cas9 nuclease) that require a combination of a trans-activating crRNA (“tracrRNA”) and a crRNA for the nuclease to cleave the target nucleotide sequence, the gRNA can be a tracrRNA:crRNA hybrid or duplex, or can be provided as a single guide RNA (sgRNA). At least 16 or 17 nucleotides of gRNA sequence corresponding to a target DNA sequence are required by Cas9 for DNA cleavage to occur. For Cas12a (Cpf1) at least 16 nucleotides of gRNA sequence corresponding to a target DNA sequence are needed to achieve detectable DNA cleavage and at least 18 nucleotides of gRNA sequence corresponding to a target DNA sequence were reported necessary for efficient DNA cleavage in vitro; see Zetsche et al. (2015) Cell, 163:759-771.

In practice, guide RNA sequences are generally designed to contain a spacer sequence of between 17-24 contiguous nucleotides (frequently 19, 20, or 21 nucleotides) with exact complementarity (e.g., perfect base-pairing) to the targeted gene or nucleic acid sequence; guide RNAs having spacers with less than 100% complementarity to the target sequence can be used (e.g., a gRNA with a spacer having a length of 20 nucleotides and between 1-4 mismatches to the target sequence), but this can increase the potential for off-target effects. The design of effective guide RNAs for use in plant genome editing is disclosed in U.S. Patent Application Publication 2015/0082478 A1. Chemically modified sgRNAs have been demonstrated to be effective in Cas9 genome editing; see, for example, Hendel et al. (2015) Nature Biotechnol., 33:985-991.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cas12a (Cpf1) endonuclease and corresponding guide RNAs and PAM sites are disclosed in U.S. Pat. No. 9,790,490. Other CRISPR nucleases useful for editing genomes include Cas 12i (see Mohanraju et al. (2016) Science, 353), Cas12b and Cas12c (see Shmakov et al. (2015) Mol. Cell, 60:385-397) and Cas12e (CasX) and Cas12d (CasY) (see Burstein et al. (2016) Nature, doi:10.1038/nature21059). For a review of diverse CRISPR systems available for genome engineering, see, e.g., Koonin et al. (2023) Biochemistry, doi: 10.1021/acs.biochem.3c00159.

Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application 61/945,700). Methods of using CRISPR technology for genome editing in plants are disclosed in US Patent Application Publications US 2015/0082478A1 and US 2015/0059010A1 and in International Patent Application PCT/US2015/038767 A1 (published as WO 2016/007347 and claiming priority to U.S. Provisional Patent Application 62/023,246).

### 1. Prime Editing

Prime editing, a variant of CRISPR/Cas gene editing, makes precise DNA sequence modifications rather than random insertions, deletions, and substitutions, thus increasing the probability of obtaining the desired effect (Anzalone et al. (2019) Nature, 576(7785): 149-157). Prime editing makes use of Cas nickases to introduce any single base pair substitution as well as small deletions or insertions into a target site. Deletions of up to 80 base pairs and insertions up to 40 bp have been produced using prime editing with a single prime editing Cas guide RNA (pegRNA) in human cells (Anzalone et al. (2019) Nature, 576: 149-157). Dual pegRNA systems are also known in the art (Choi et al. (2021) Nature Biotechnology, 40(2): 218-226; Lin et al. (2021) Nature Biotechnology, 39(8): 923-927) and can be used to generate precise large deletions, or to improve editing efficiency for small insertions, deletions, or substitutions. Additionally, dual pegRNA systems where the extension of the pegRNAs are not complementary to the endogenous locus, but are complementary to one another, can be used to replace endogenous sequences and/or mediate larger insertions (Anzalone et al. (2022) Nature Biotechnology, 40(5): 731-740).

Prime editing can also be accomplished with Cas nucleases in place of Cas nickases (Adikusuma et al. (2021) Nucleic Acids Res., 49(18): 10785-10795). In some embodiments, prime editing uses (i) a Cas nuclease, optionally a Cas9 or Cas12 nuclease, fused to a reverse transcriptase (Cas-RT), optionally a M-MLV reverse transcriptase, and (ii) a pegRNA that both specifies the genome target site and has an extension that encodes the target edit within a template for the reverse transcriptase. In some embodiments, the binding of the pegRNA directs the Cas nuclease to create a double-stranded break in the DNA at the target site. The extension of the pegRNA binds to the cut DNA that has an exposed 3′-hydroxyl group, priming the reverse transcriptase to produce a DNA strand that is complementary to the extension of the pegRNA. This DNA strand will include the complement to any desired edits present in the provided pegRNA extension. Mismatch repair by the cell will then resolve the mismatch between the unedited parent strand and the edited product of the reverse transcriptase, thus introducing the desired edits into the genome. Prime editing systems may also include elements to inhibit mismatch repair, or to nick the unedited parent strand to increase editing efficiency.

### 2. Precise Base Editing

Targeted modification of sequences may also be accomplished using precise base editing (PBE), another recently developed variant of the CRISPR/Cas gene editing system. Precise base editing enables accurate single-base editing of a genome using a fusion protein comprising a nuclease dead Cas9 (dCas9) protein and a cytidine deaminase. A dCas9 protein contains mutations in the endonuclease domains (e.g., in the HNH subdomain and/or RuvC subdomain of the DNA cleavage domain) but retains gRNA-directed DNA-binding ability, while the cytidine deaminase can catalyze deamination of cytidine (C) on DNA to form uracil (U). Thus, a guide RNA can direct a dCas9-cytidine deaminase fusion protein to a target sequence in the plant genome. Due to the absence of the Cas9 nuclease activity, the DNA double strand is not cleaved. The deaminase domain in the fusion protein converts the cytidine (C) of the single-stranded DNA, produced in the formation of the Cas9-gRNA-DNA complex, to uracil (U) and the substitution of C to T is achieved by base mismatch repair. The precise base editor system suitable for use in the present invention includes, but is not limited to, the system described in Zong et al. (2017) Nature Biotechnology 35(5): 438-440.

### B. Zinc Finger Nucleases

Zinc finger nucleases (ZFNs) are engineered proteins comprising a zinc finger DNA-binding domain fused to a nucleic acid cleavage domain, e.g., a nuclease. The zinc finger binding domains provide specificity and can be engineered to specifically recognize any desired target DNA sequence. For a review of the construction and use of ZFNs in plants and other organisms, see, e.g., Urnov et al. (2010) Nature Rev. Genet., 11:636-646. The zinc finger DNA binding domains are derived from the DNA-binding domain of a large class of eukaryotic transcription factors called zinc finger proteins (ZFPs). The DNA-binding domain of ZFPs typically contains a tandem array of at least three zinc “fingers” each recognizing a specific triplet of DNA. A number of strategies can be used to design the binding specificity of the zinc finger binding domain. One approach, termed “modular assembly”, relies on the functional autonomy of individual zinc fingers with DNA. In this approach, a given sequence is targeted by identifying zinc fingers for each component triplet in the sequence and linking them into a multifinger peptide. Several alternative strategies for designing zinc finger DNA binding domains have also been developed. These methods are designed to accommodate the ability of zinc fingers to contact neighboring fingers as well as nucleotide bases outside their target triplet. Typically, the engineered zinc finger DNA binding domain has a novel binding specificity, compared to a naturally occurring zinc finger protein.

Zinc finger engineering methods include, for example, rational design and various types of selection. Rational design includes, for example, the use of databases of triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, e.g., U.S. Pat. Nos. 6,453,242 and 6,534,261. Exemplary selection methods (e.g., phage display and yeast two-hybrid systems) are well known and described in the literature. In addition, enhancement of binding specificity for zinc finger binding domains has been described in U.S. Pat. No. 6,794,136. In addition, individual zinc finger domains may be linked together using any suitable linker sequences. Examples of linker sequences are publicly known, see, e.g., U.S. Pat. Nos. 6,479,626; 6,903,185; and 7,153,949.

The nucleic acid cleavage domain is non-specific and is typically a restriction endonuclease, such as Fokl. This endonuclease must dimerize to cleave DNA. Thus, cleavage by Fokl as part of a ZFN requires two adjacent and independent binding events, which must occur in both the correct orientation and with appropriate spacing to permit dimer formation. The requirement for two DNA binding events enables more specific targeting of long and potentially unique recognition sites. Fokl variants with enhanced activities have been described; see, e.g., Guo et al. (2010) J. Mol. Biol., 400:96-107.

### C. Talens

Transcription activator like effectors (TALEs) are proteins secreted by certain Xanthomonas species to modulate gene expression in host plants and to facilitate the colonization by and survival of the bacterium. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site has been found. The simple relationship between amino acid sequence and DNA recognition of the TALE binding domain allows for the design of DNA binding domains of any desired specificity. TALEs can be linked to a non-specific DNA cleavage domain (e.g., a nuclease) to prepare sequence-specific endonucleases referred to as TAL-effector nucleases or TALENs. As in the case of ZFNs, a restriction endonuclease, such as Fokl, can be conveniently used. For a description of the use of TALENs in plants, see Mahfouz et al. (2011) Proc. Natl. Acad. Sci. USA, 108:2623-2628 and Mahfouz (2011) GM Crops, 2:99-103.

### D. Meganucleases

Meganucleases are sequence-specific endonucleases with large (>14 bp) cleavage sites that can deliver DNA double-strand breaks (DSBs) at specific loci in living cells (Thierry and Dujon (1992) Nucleic Acids Res., 20, 5625-5631). Meganucleases have been used to stimulate homologous recombination in the vicinity of their target sequences in cultured cells and plants (Rouet et al. (1994)Mol. Cell. Biol., 14, 8096-106; Choulika et al. (1995)Mol. Cell. Biol., 15, 1968-73; Donoho et al. (1998) Mol. Cell. Biol., 18, 4070-8; Elliott et al. (1998) Mol. Cell. Biol., 18, 93-101; Sargent et al. (1997) Mol. Cell. Biol., 17, 267-77; Puchta et al. (1996) Proc. Natl. Acad. Sci., 93, 5055-60; and Chiurazzi et al. (1996) Plant Cell, 8, 2057-2066). In nature, meganucleases are essentially represented by homing endonucleases (HEs), a family of endonucleases encoded by mobile genetic elements whose function is to initiate DNA double-strand break (DSB)-induced recombination events in a process referred to as “homing” (Chevalier and Stoddard (2001) Nucleic Acids Res., 29, 3757-74; Kostriken et al. (1983) Cell, 35, 167-74; and Jacquier and Dujon (1985), Cell, 41, 383-94).

### E. Homology Directed Repair (HDR)

The insertion of desired sequences at a genomic target site may be accomplished through homology-directed repair (HDR). HDR is a genome editing method that can be used for precise insertion of a sequence from a provided DNA donor template at a specified site in genomic DNA. As used herein, the term “donor template” refers to a DNA construct that includes a polynucleotide of interest to be inserted into the target site of a gene editing system, as well as potentially including a first and a second region of homology that flank the polynucleotide of interest.

Donor template DNA molecules include DNA molecules comprising, from 5′ to 3′, a first homology arm, at least one replacement DNA sequence (i.e., the desired sequence), and a second homology arm. The homology arms contain sequences that are partially or completely homologous to genomic DNA (gDNA) sequences flanking an endonuclease recognition sequence in the gDNA (e.g., a genomic target site). The replacement DNA can comprise an insertion, deletion, or substitution of 1 or more DNA base pairs relative to the target gDNA. In certain embodiments, a donor DNA template homology arm is about 20, 50, 100, 200, 400, or 600 to about 800, or 1000 base pairs in length. Donor DNA templates can be synthesized either chemically or enzymatically (e.g., in a polymerase chain reaction (PCR)).

In certain embodiments, a donor template DNA molecule is delivered to a eukaryotic cell (e.g., a plant cell) in a circular (e.g., a plasmid or a viral vector including a gemini virus vector) or a linear DNA molecule. In certain embodiments, a circular or linear DNA molecule that is used can comprise a modified donor template DNA molecule comprising, from 5′ to 3′: a first copy of an endonuclease recognition sequence, the first homology arm, the replacement DNA, the second homology arm, and a second copy of the endonuclease recognition sequence. In some embodiments, an RNA template of a reverse transcriptase is provided. In some embodiments, a reverse transcriptase is provided in addition to an RNA. In some embodiments, the method comprises use of a single stranded DNA donor template. In some embodiments, a single or double stranded RNA template is used. In some embodiments, the method comprises use of a DNA/RNA hybrid.

### III. PLANT TRANSFORMATION METHODS

As used herein, the terms “transformation”, “introduction” or “delivery” refers to the transfer of an exogenous polynucleotide into a host cell, irrespective of the method used for transfer. As used herein, the terms “plasmid”, “vector” and “cassette” refer to an extra-chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell.

Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art.

As used herein, the term “plant” includes a whole plant and any descendant, cell, tissue, or part of a plant. Any part(s) of a plant include, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, silk, flowers, kernels, ears, cobs, husks, or stalks.

### A. Transformation Methods

Numerous methods for plant transformation have been developed including biological and physical plant transformation protocols. See, for example, Miki et al., “Procedures for Introducing Foreign DNA into Plants,” in Methods in Plant Molecular Biology and Biotechnology, Glick and 65 Thompson Eds., CRC Press, Inc., Boca Raton, pp. 67-88 (1993). In addition, expression vectors and in vitro culture methods for plant cell or tissue transformation and regeneration of plants are available. See, for example, Gruber et al., “Vectors for Plant Transformation,” in Methods in Plant Molecular Biology and Biotechnology, Glick and Thompson Eds., CRC Press, Inc., Boca Raton, pp. 89-119 (1993). In certain embodiments, one or more methods are employed to deliver the gene editing system (e.g., comprising a polynucleotide, polypeptide or combination thereof) into a plant or plant part (e.g., through barriers such as a cell wall, a plasma membrane, a nuclear envelope, and/or other lipid bilayer).

One method for introducing an expression vector into plants is based on the natural transformation system of Agrobacterium. See, for example, Horsch et al. (1985) Science, 227:1229. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I. (1991) Crit. Rev. Plant Sci., 10:1. Descriptions of Agrobacterium vector systems and methods for Agrobacterium mediated gene transfer are provided by Gruber et al., supra, Miki et al., supra, and Moloney et al. (1989) Plant Cell Reports, 8:238. See also, U.S. Pat. No. 5,563,055 issued Oct. 8, 1996.

Several methods of plant transformation, collectively referred to as direct gene transfer, have been developed as an alternative to Agrobacterium mediated transformation. A generally applicable method of plant transformation is microprojectile-mediated transformation where DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. See, for example, Sanford et al. (1987) Part. Sci. Technol., 5:27; Sanford, J. C. (1988) Trends Biotech., 6:299; Klein et al. (1988) Bio Tech., 6:559-563; Sanford, J. C. (1990) Physiol Plant, 7:206; and Klein et al. (1992) Biotechnology, 10:268. See also, U.S. Pat. No. 5,015,580 issued May 14, 1991 and U.S. Pat. No. 5,322,783 issued Jun. 21, 1994.

Another method for physical delivery of DNA to plants is sonication of target cells. See, for example, Zhang et al. (1991) Bio Technology, 9:996. Alternatively, liposome and spheroplast fusion have been used to introduce expression vectors into plants. See, for example, Deshayes et al. (1985) EMBO J., 4:2731; Christou et al. (1987) Proc Nat/. Acad. Sci., 84:3962. Direct uptake of DNA into protoplasts using CaCl₂ precipitation, polyvinyl alcohol or poly-L-ornithine have also been reported (Hain et al. (1985) Mal. Gen. Genet., 199:161 and Draper et al. (1982) Plant Cell Physiol., 23:451). Electroporation of protoplasts and whole cells and tissues have also been described (Donn et al. (1990) In Abstracts of VII'th International Congress on Plant Cell and Tissue Culture IAPTC, A2-38, p. 53; D'Halluin et al. (1992) Plant Cell, 4:1495-1505; and Spencer et al. (1994) Plant Mol. Biol., 24:51-61).

In certain embodiments, a polynucleotide-, polypeptide-, or RNP (ribonucleoprotein)-containing composition are delivered directly, for example by direct contact of the composition with a plant tissue or cell. Compositions can be provided in the form of a liquid, a solution, a suspension, an emulsion, a reverse emulsion, a colloid, a dispersion, a gel, liposomes, micelles, an injectable material, an aerosol, a solid, a powder, a particulate, a nanoparticle, or a combination thereof. Compositions can be applied directly to a plant, plant part, plant cell, or plant explant (e.g., through abrasion or puncture or other-wise disruption of the cell wall or cell membrane, by spraying or dipping or soaking or otherwise directly contacting, by microinjection). For example, a plant cell or plant protoplast is soaked in a liquid composition comprising the gene editing system, whereby the gene editing system is delivered to the plant cell.

In certain embodiments, the gene editing composition is delivered using negative or positive pressure, for example, using vacuum infiltration or application of hydrodynamic or fluid pressure. In certain embodiments, the gene editing composition is introduced into a plant cell or plant protoplast, e.g., by microinjection or by disruption or deformation of the cell wall or cell membrane, for example by physical treatments such as by application of shear forces, or treatment with a chemical or physical delivery agent such as surfactants, liposomes, or nanoparticles; see, e.g., delivery of materials to cells employing microfluidic flow through a cell-deforming constriction as described in US Published Patent Application 2014/0287509.

Other techniques useful for delivering the gene editing composition to a plant cell or plant protoplast include: ultrasound or sonication; vibration, friction, shear stress, vortexing, cavitation; centrifugation or application of mechanical force; mechanical cell wall or cell membrane deformation or breakage; enzymatic cell wall or cell membrane breakage or permeabilization; abrasion or mechanical scarification (e.g., abrasion with carborundum or other particulate abrasive or scarification with a file or sandpaper) or chemical scarification (e.g., treatment with an acid or caustic agent); and electroporation.

In certain embodiments, the gene editing composition is provided by bacterially mediated (e.g., Agrobacterium sp., Rhizobium sp., Sinorhizobium sp., Mesorhizobium sp., Bradyrhizobium sp., Azobacter sp., Phyllobacterium sp.) transfection of the plant cell or plant protoplast with a polynucleotide encoding the gene editing system; see, e.g., Broothaerts et al. (2005) Nature, 433:629-633.

Techniques for effecting genome editing in crop plants (e.g., maize), include use of morphogenic factors such as Wuschel (WUS), Ovule Development Protein (ODP), and/or Babyboom (BBM) which can improve the efficiency of recovering plants with desired genome edits. In some aspects, the morphogenic factor comprises WUS1, WUS2, WUS3, WOX2A, WOX4, WOX5, WOX9, BBM2, BMN2, BMN3, and/or ODP2. In certain embodiments, compositions and methods for using WUS, BBM, and/or ODP, as well as other techniques which can be adapted for effecting genome edits in plants, are set forth in US 20030082813, US 20080134353, US 20090328252, US 20100100981, US 20110165679, US 20140157453, US 20140173775, and US 20170240911.

In certain embodiments, the gene edits can be effected in regenerable plant parts (e.g., plant embryos) of crop plants by transient provision of a gene editing system or polynucleotides encoding the same and do not necessarily require incorporating a selectable marker gene into the plant genome (e.g., US 20160208271 and US 20180273960; see also Svitashev et al. (2016) Nat Commun., 7:13274). In certain embodiments, the genome alteration is limited only to those cells from which DNA is inherited in subsequent generations, which is advantageous where it is desirable that expression of the genome-editing system be limited to avoid genotoxicity or other unwanted effects.

### B. Expression Vectors

Plant transformation may involve the construction of an expression vector which will function in plant cells. Such a vector can comprise DNA encoding a gene under control of, or operatively linked to, a regulatory element (for example, a promoter). The expression vector may contain one or more such operably linked gene/regulatory element combinations. The vector(s) may be in the form of a plasmid and can be used alone or in combination with other plasmids to incorporate transgenes into the genetic material of a plant using the transformation methods described above.

### 1. Selectable Markers

Expression vectors include at least one genetic marker operably linked to a regulatory element (for example, a promoter) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art (e.g., green fluorescent protein (GFP) or beta-glucuronidase (gus).

In some embodiments, an expression vector comprising a gene editing system, such as a CRISPR/Cas gene editing system, includes a selectable marker gene for screening or selection of cells or plants expressing the vector. Selectable markers include genes that confer resistance to herbicidal compounds, such as glyphosate, sulfonylureas, glufosinate ammonium, bromoxynil, imidazolinones, and 2,4-dichlorophenoxyacetate (2,4-D). Such selectable marker genes and selective agents include the maize HRA gene (Lee et al. (1988) EMBO J, 7:1241-1248) which confers resistance to sulfonylureas and imidazolinones; the CP4 gene that confers resistance to glyphosate (US Reissue Patent RE039247); the GAT gene which confers resistance to glyphosate (Castle et al. (2004) Science, 304:1151-1154); genes that confer resistance to spectinomycin such as the aadA gene (Svab et al. (1990) Plant Mol Biol. 14:197-205); the bar gene that confers resistance to glufosinate ammonium (White et al. (1990) Nucl. Acids Res. 25:1062); PAT, or moPAT for corn (see Rasco-Gaunt et al. (2003) Plant Cell Rep. 21:569-76; also see Sivamani et al. (2019)); and the PMI gene that permits growth on mannose-containing medium (Negrotto et al. (2000) Plant Cell Rep. 22:684-690).

In certain embodiments, a counter-selectable marker can be used. Such counter-selectable markers can in certain embodiments be incorporated into any DNA that is not intended for insertion into a host cell genome at target editing sites. In such embodiments, non-limiting examples of DNAs with counter-selectable markers include any DNA molecules that are linked to DNAs encoding HDR-promoting agents (e.g., SSB, SSAP, and/or exonucleases), gene-editing molecules, and/or donor template DNA molecules. In certain embodiments, the counter-selectable marker is linked to the donor template DNA and optionally separated from the donor template DNA by a target site sequence. Examples of counter-selectable markers that can be used in plants include cytosine deaminase genes (e.g., used in conjunction with 5-fluorocytosine; Schlaman and Hooykaas, 1997), phosphonate ester hydrolases (e.g., used in conjunction with phosphonate esters of glyphosate including glycerol glyphosate; Dotson, et al. 1996), and a nitrate reductase (e.g., used in conjunction with chlorate on media containing ammonia as a sole nitrogen source; Nussaume, et al. 1991).

### 2. Promoters

Genes included in expression vectors must be driven by a nucleotide sequence comprising a regulatory element (for example, a promoter). Several types of promoters are well known in the transformation arts as are other regulatory elements that can be used alone or in combination with promoters. As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Tissue-specific, tissue-preferred (i.e., developmentally controlled), cell-type specific, and inducible promoters constitute the class of “non-constitutive” promoters. A “constitutive” promoter is a promoter that is active under most environmental conditions.

Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters that initiate transcription only in a certain tissue are referred to as “tissue-specific.” A “cell-type specific” promoter primarily drives expression in certain cell types in one or more organs, for example, vascular cells in roots or leaves. An “inducible” promoter is a promoter which is under environmental control. Examples of environmental conditions that may affect transcription by inducible promoters include anaerobic conditions or the presence of light.

In some embodiments, one or more expression vectors are constructed comprising polynucleotides that encode a gene editing system, such as a CRISPR/Cas gene editing system comprising a guide RNA and Cas nuclease. In some embodiments, the one or more polynucleotides encoding the gene editing system are operably linked to a promoter. In some embodiments, the polynucleotides encoding the gene editing system are operably linked to different promoters. In some embodiments, the polynucleotides encoding the gene editing system are operably linked to a constitutive, inducible, tissue-specific or developmental promoter that drives expression in plants.

In certain embodiments, the promoter operably linked to one or more polynucleotides is a constitutive promoter that drives gene expression in plant cells. Examples of constitutive promoters for use in plants include a CaMV 35S promoter as disclosed in U.S. Pat. Nos. 5,858,742 and 5,322,938, a rice actin promoter as disclosed in U.S. Pat. No. 5,641,876, a maize chloroplast aldolase promoter as disclosed in U.S. Pat. No. 7,151,204, and the nopaline synthase (NOS) and octopine synthase (OCS) promoters from Agrobacterium tumefaciens. In some embodiments, the promoter is selected from the group consisting of CaMV35S, ubiquitin, Rsyn7, NOS, MAS, ALS, pEMU, AtU3, AtU6, OsU3, OsU6, Pol II, Pol III, a tissue-specific promoter, and a cell-specific type promoter.

Other contemplated promoters include cell-specific or tissue-specific or developmentally regulated promoters, for example, a promoter that limits the expression of the gene editing system to germline or reproductive cells (e.g., promoters of genes encoding DNA ligases, recombinases, replicases, or other genes specifically expressed in germline or reproductive cells). Developmentally regulated promoters that can be used in plant cells include Phospholipid Transfer Protein (PLTP), fructose-1,6-bisphosphatase protein, NAD(P)-binding Rossmann-Fold protein, adipocyte plasma membrane-associated protein-like protein, Rieske [2Fe-2S] iron-sulfur domain protein, chlororespiratory reduction 6 protein, D-glycerate 3-kinase, chloroplastic-like protein, chlorophyll a-b binding protein 7, chloroplastic-like protein, ultraviolet-B-repressible protein, Soul heme-binding family protein, Photosystem I reaction center subunit psi-N protein, and short-chain dehydrogenase/reductase protein. These promoters are disclosed in US Patent Application Publication No. 20170121722. In certain embodiments, the promoter operably linked to one or more polynucleotides encoding the gene editing system is a promoter from figwort mosaic virus (FMV), a RUBISCO promoter, or a pyruvate phosphate dikinase (PPDK) promoter, which is active in photosynthetic tissues.

### 3. Additional Elements

Expression vectors or polynucleotides provided herein may contain a DNA segment near the 3′ end of an expression cassette that acts as a signal to terminate transcription and directs polyadenylation of the resultant mRNA and may also support promoter activity. Such a 3′ element is commonly referred to as a “3′-untranslated region” or “3′-UTR” or a “polyadenylation signal.” In some cases, plant gene-based 3′ elements (or terminators) consist of both the 3′-UTR and downstream non-transcribed sequence (Nuccio et al., 2015). Useful 3′ elements include: Agrobacterium tumefaciens nos 3′, tml 3′, tmr 3′, tms 3′, ocs 3′, and tr7 3′ elements disclosed in U.S. Pat. No. 6,090,627 and 3′ elements from plant genes such as the heat shock protein 17, ubiquitin, and fructose-1,6-biphosphatase genes from wheat (Triticum aestivum), and the glutelin, lactate dehydrogenase, and beta-tubulin genes from rice (Oryza sativa), disclosed in US Patent Application Publication 2002/0192813 A1.

In some embodiments, expression vectors include additional elements for improving delivery to a plant cell or plant protoplast, or for directing or modifying expression of one or more gene editing system elements. For example, fusing a sequence encoding a cell-penetrating peptide, localization signal, transit, or targeting peptide to a Cas endonuclease, or adding a nucleotide sequence to stabilize a guide RNA. In some embodiments, multiple expression vectors are delivered to the plant, plant part or plurality of plants.

### IV. PLANT BREEDING

The improvement of agronomic traits in a crop plant is a method of improving germplasm or general crop populations resulting in an elite crop plant. As used herein, the phrase “elite crop plant” refers to a plant which has undergone breeding to provide one or more desirable traits for human benefit. Elite crop plants are typically homozygous, e.g. inbred or doubled haploid, and exhibit desirable agronomic traits such as high yield, good quality and resistance to biotic or abiotic stresses. Elite crop plants can include inbred lines which are selfed to produce non-hybrid cultivars or varieties, or to produce pollen donor or recipient lines for hybrid seed production (e.g., to produce F1 hybrid plants). Elite crop plants can include hybrid F1 progeny of a cross between two distinct elite inbred or doubled haploid plant lines.

However, the gene pools of elite crop plants exhibit limited functional diversity which creates a bottleneck for further improvement through breeding. In addition, elite crop plants are notoriously difficult to directly transform. As a result, model cultivars or varieties that are more amenable to transformation but may lack the qualities of elite lines (e.g., have poor yield or quality) are typically used for the creation of transgenic and/or edited plant lines. A transgene or edited gene is typically introduced into an elite variety using traditional backcrossing techniques that are well known in the plant breeding arts. For example, a backcrossing approach could be used to move an engineered trait from a public, non-elite cultivar into an elite cultivar, or from a cultivar containing a foreign gene in its genome into a cultivar or cultivars that do not contain that gene. As used herein, “crossing” can refer to a simple X by Y cross or the process of backcrossing depending on the context.

The term “backcrossing” as used herein refers to the repeated crossing of a hybrid progeny back to the recurrent parent, i.e., backcrossing 1, 2, 3, 4, 5, 6, 7, 8, or more times to the recurrent parent. The parental plant that contributes the gene for the desired characteristic is termed the nonrecurrent or donor parent (e.g., the transgenic and/or edited non-elite cultivar). This terminology refers to the fact that the nonrecurrent parent is used one time in the backcross protocol and therefore does not recur. The parental plant to which the gene or genes from the nonrecurrent parent are transferred is known as the recurrent parent (e.g., the elite cultivar) as it is used for several rounds in the backcrossing protocol (Poehlman & Sleper (1994); Fehr, Principles of Cultivar Development, pp. 261-286 (1987)).

In a typical backcross protocol, the original cultivar of interest (recurrent parent) is crossed to a second cultivar (nonrecurrent parent) that carries the single gene of interest to be transferred. The resulting progeny from this cross are then crossed again to the recurrent parent and the process is repeated until a plant is obtained wherein essentially all of the morphological and physiological characteristics of the recurrent parent are recovered, in addition to the single transferred gene (e.g., a transgene or edited gene) from the nonrecurrent parent. Backcrosses can be repeated and/or supplemented by molecular assisted breeding techniques using SNP or other nucleic acid markers to select for recurrent parent germplasm until a desired recurrent parent percentage is obtained (e.g., at least about 95%, 96%, 97%, 98%, or 99% recurrent parent percentage).

### V. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as well as necessarily defines the exact complements, as is known to one of ordinary skill in the art. Where a term is provided in the singular, the inventors also contemplate embodiments described by the plural of that term.

As used herein, the term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features to which they refer while not excluding any additional unspecified features.

As used herein, the phrase “plant cell” can refer either a plant cell having a plant cell wall or to a plant cell protoplast lacking a plant cell wall.

As used herein, the term “plant” includes a whole plant and any descendent, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, a protoplast, callus, or another group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype of the plant.

Agronomic trait. A trait deemed beneficial to crop plants, such as traits related to plant growth, crop maintenance, and the consumption or utilization of the crop plant.

Allele. Any of one or more alternative forms of a genetic sequence. In a diploid cell or organism, the two alleles of a given sequence typically occupy corresponding loci on a pair of homologous chromosomes.

Allelic series. A range of phenotypes observable due to variable mutations across multiple alleles for at least one given gene.

Allelic variant. A polynucleotide or polypeptide sequence variant that occurs in a different strain, variety, or isolate of a given organism.

Backcrossing. A process in which a breeder crosses progeny back to one of the parental genotypes one or more times. Commonly used to introduce one or more locus conversions from one genetic background into another.

Conserved sequence. A nucleotide or amino acid sequence that is identical or similar across a variety of species or organisms.

In-frame deletion. A deletion of a multiple of three nucleotides that removes one or more amino acid codons within a protein coding sequence.

Operably linked. A juxtaposition wherein the components so described are in a relationship permitting them to function in their intended manner. For instance, a promoter is operably linked to a coding sequence if the promoter affects its transcription or expression.

Orthologous. Genes, or proteins encoded by those genes, that are from different species but which have the same function (e.g., encode enzymes that catalyze the same reactions or encode transcriptional regulators that control expression of genes with similar functions. Orthologous genes will typically encode proteins with some degree of sequence identity (e.g., at least 40%, 50%, 60%, 70%, 80%, 90%, or 95% sequence identity), conservation of sequence motifs, and/or conservation of structural features.

Promoter. A region of DNA upstream from the start of transcription and involved in recognition and binding of RNA polymerase and other proteins to initiate transcription. A “plant promoter” is a promoter capable of initiating transcription in plant cells.

Regeneration. The development of a plant from tissue culture.

### VI. EXEMPLARY EMBODIMENTS

Among the provided embodiments are:
1. A method of modulating the activity of a protein of interest in a plant or plant part comprising providing a Cas nuclease and a guide RNA for the Cas nuclease to the plant or plant part,
wherein the guide RNA is complementary to a target sequence in a target site,
wherein the target site comprises one or more codons of a plant gene encoding a protein of interest,
wherein editing by the Cas nuclease and the guide RNA results in deletion of one or more amino acids in the protein of interest.
2. A method of creating an allelic series of a plant gene comprising providing a Cas nuclease and a guide RNA for the Cas nuclease to each of a plurality of plants,
wherein the guide RNA is complementary to a target sequence in a target site,
wherein the target site comprises one or more codons of a plant gene encoding a protein of interest,
wherein editing by the Cas nuclease and the guide RNA generates a series of different amino acid deletions in the protein of interest across the plurality of plants.
3. The method of embodiment 2, further comprising selecting progeny of the plurality of plants,
wherein each of the progeny contain different amino acid deletions in the protein of interest.
4. The method of embodiment 3, wherein the selected progeny exhibit a range of phenotypes.
5. The method of embodiment 4, wherein the range of phenotypes is a spectrum of intermediate phenotypes for a quantitative agronomic trait.
6. The method of embodiment 4 or embodiment 5, wherein one or more of the selected progeny exhibits an improvement in an agronomic trait.
7. The method of embodiment 1, further comprising selecting a progeny from the plant,
wherein the progeny exhibits an improvement in an agronomic trait due to the deletion of one or more amino acids in the protein of interest.
8. The method of any of embodiments 3-7, further comprising backcrossing the selected progeny to remove the Cas nuclease and the guide RNA.
9. The method of any of embodiments 3-8, wherein the selected progeny no longer contains the Cas nuclease and the guide RNA.
10. The method of any one of embodiments 2-9, wherein the method comprises providing two or more, three or more, four or more, five or more, six or more, seven or more, or eight or more guide RNAs to the plurality of plants.
11. The method of embodiment 10, wherein each of the guide RNAs targets one or more target sites within a single exon of the plant gene encoding the protein of interest.
12. The method of embodiment 10, wherein each of the guide RNAs targets one or more target sites in different exons of the plant gene encoding the protein of interest.
13. The method of any of embodiments 1-12, wherein the guide RNA targets an evolutionary conserved sequence in the protein of interest.
14. The method of any of embodiments 1-13, wherein the deletion of one or more amino acids in the protein of interest comprises an in-frame deletion.
15. The method of embodiment 14, wherein the in-frame deletion comprises a deletion of a multiple of 3 nucleotides that results in the deletion of one or more codons of the plant gene encoding the protein of interest.
16. The method of embodiment 15, wherein the in-frame deletion further comprises a partial deletion of one codon and a synonymous mutation in an adjacent codon.
17. The method of embodiment 14, embodiment 15, or embodiment 16, wherein the in-frame deletion creates an allelic variant of the plant gene encoding the protein of interest.
18. The method of any of embodiments 14-17, wherein the in-frame deletion comprises a deletion of two, three, four, five, six, seven, eight, nine, or ten codons from the plant gene encoding the protein of interest.
19. The method of any of embodiments 14-18, wherein the in-frame deletion comprises a deletion of one or more codons encoding cysteine from the plant gene encoding the protein of interest.
20. The method of any of clams 14-19, wherein the in-frame deletion comprises a deletion of a stop codon from the plant gene encoding the protein of interest.
21. The method of embodiment 20, wherein deletion of the stop codon results in an increase in the length of the protein of interest.
22. The method of any of embodiments 14-21, wherein the in-frame deletion comprises a deletion of three, six, nine, 12, 15, 18, 21, 24, 27, or 30 nucleotides comprising one or more codons from the plant gene encoding the protein of interest.
23. The method of any of embodiments 1-22, wherein editing by the Cas nuclease and the guide RNA increases or reduces the biological activity of the protein of interest.
24. The method of any of embodiments 1-23, wherein editing by the Cas nuclease and the guide RNA results in a different structural isoform of the protein of interest.
25. The method of any of embodiments 1-24, wherein editing by the Cas nuclease and the guide RNA results in truncation of the protein of interest.
26. The method of any of embodiments 1-25, wherein editing by the Cas nuclease and the guide RNA results in a N-terminal or C-terminal truncation of the protein of interest.
27. The method of any of embodiments 1-26, wherein editing by the Cas nuclease and the guide RNA removes a site of a post-translational modification or co-translational modification from the protein of interest.
28. The method of any of embodiments 1-27, wherein editing by the Cas nuclease and the guide RNA results in decreased post-translational modification or co-translational modification of the protein of interest.
29. The method of any of embodiments 1-28, wherein editing by the Cas nuclease and the guide RNA increases turnover of the protein of interest.
30. The method of any of embodiments 1-28, wherein editing by the Cas nuclease and the guide RNA decreases turnover of the protein of interest.
31. The method of any of embodiments 1-30, wherein editing by the Cas nuclease and the guide RNA stabilizes an mRNA transcript encoding the protein of interest.
32. The method of any of embodiments 1-30, wherein editing by the Cas nuclease and the guide RNA destabilizes an mRNA transcript encoding the protein of interest.
33. The method of any of embodiments 1-32, wherein editing by the Cas nuclease and the guide RNA results in alternative splicing of an mRNA transcript encoding the protein of interest.
34. The method of any of embodiments 1-33, wherein editing by the Cas nuclease and the guide RNA removes a binding site on the protein of interest.
35. The method of embodiment 34, wherein the binding site removed from the protein of interest is for a protein, nucleic acid, lipid, carbohydrate or small molecule.
36. The method of any of embodiments 1-35, wherein editing by the Cas nuclease and the guide RNA alters the subcellular localization of the protein of interest.
37. The method of any of embodiments 1-36, wherein editing by the Cas nuclease and the guide RNA alters the intercellular transport of the protein of interest.
38. The method of any of embodiments 1-37, wherein editing by the Cas nuclease and the guide RNA decreases the level of the protein of interest.
39. The method of embodiment 38, wherein the level of the protein of interest is decreased by at least 1%, at least 5%, at least 10%, at least 15%, at least 20%, at least 30%, at least 40%, at least 50%, at least 70%, at least 80%, or at least 90%.
40. The method of any of embodiments 1-39, wherein editing by the Cas nuclease and the guide RNA increases the level of the protein of interest.
41. The method of embodiment 40, wherein the level of the protein of interest is increased by at least 1%, at least 5%, at least 10%, at least 15%, at least 20%, at least 30%, at least 40%, at least 50%, at least 70%, at least 80%, at least 90%, at least 100%, at least 200%, at least 300%, at least 400%, at least 500%, at least 600%, at least 700%, at least 800%, or up to 900%.
42. The method of any of embodiments 1-41, wherein the Cas nuclease is a Cas9, Cas12a (Cpf1), Cas12c, Cas12d, Cas12e, Cas12b, Cas12h, Cas12i, Cas12j, Cas12f, or an engineered Cas nuclease.
43. The method of any of embodiments 1-42, wherein the Cas nuclease is a Cas12i nuclease.
44. The method of embodiment 43, wherein the Cas12i nuclease is derived from Lachnospiraceae.
45. The method of any of embodiments 1-42, wherein the Cas nuclease is a Cas12a (Cpf1) nuclease.
46. The method of embodiment 43, wherein the Cas12a nuclease is derived from Francisella novicida, Acidaminococcus sp, or Lachnospiraceae.
47. The method of any of embodiments 1-46, wherein expression of the Cas nuclease is driven by a constitutive, inducible, tissue-specific or developmental promoter.
48. The method of any of embodiments 1-47, wherein the Cas nuclease is codon-optimized for expression in dicots or monocots.
49. The method of any of embodiments 1-48, wherein the Cas nuclease and the guide RNA are provided by particle bombardment of a plant, plant part or plurality of plants.
50. The method of any of embodiments 1-49, wherein the Cas nuclease and the guide RNA are provided by Agrobacterium-mediated transformation of the plant, plant part or plurality of plants.
51. The method of any of embodiments 1-50, wherein the guide RNA is provided by application of a composition comprising the guide RNA to the plant, plant part or plurality of plants.
52. The method of any of the embodiments 1-51, further comprising providing a donor template.
53. The method of embodiment 52, wherein insertion of the donor template results in the precise deletion of one or more codons in the plant gene encoding the protein of interest.
54. The method of any of embodiments 1-53, wherein the Cas nuclease is associated with a reverse transcriptase.
55. The method of any of embodiments 5-54, wherein the agronomic trait is selected from the group consisting of herbicide tolerance, disease resistance, insect or pest resistance, increased grain yield, increased oil, altered plant maturity, enhanced stress tolerance, and altered morphological characteristics.
56. The method of any of embodiments 1-55, wherein the plant gene encoding the protein of interest is a developmental gene.
57. The method of any of embodiments 1-56, wherein the plant gene encoding the protein of interest exhibits dosage sensitivity.
58. The method of any of embodiments 1-57, wherein the plant gene encoding the protein of interest regulates the cell cycle, cell growth, stem cell fate or hormone signaling.
59. The method of any of embodiments 1-58, wherein the protein of interest is a signaling peptide or transcription factor.
60. The method of any of embodiments 1-59, wherein low or high levels of the protein of interest are associated with an agronomically undesirable phenotype.
61. The method of any of embodiments 1-60, wherein the protein of interest is involved in drought tolerance, disease resistance, pest resistance, stress tolerance, yield, shape, odor, texture, metabolite production, pigmentation, seed fecundity, endoreduplication, sugar content, pH, improved shelf life or storability, cell differentiation, branching, plant height, time to fruit set, or light reception.
62. The method of any of embodiments 1-61, wherein the plant or plurality of plants is selected from the group consisting of maize, rice, sorghum, rye, barley, wheat, pearl millet, foxtail millet, proso millet, fonio millet, teff, flax, oats, sugarcane, turfgrass, switchgrass, soybean, canola, alfalfa, sunflower, cotton, tobacco, tomato, peanut, potato, cannabis, a forage crop, an industrial crop, a woody crop, a biomass crop, and Arabidopsis.
63. The method of any of embodiments 1-62, wherein the plant part is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.
64. The method of any of embodiments 1-63, wherein the plant, plant part or plurality of plants is a crop plant.
65. The method of any of embodiments 1-64, wherein the plant, plant part or plurality of plants is a monocot or a dicot.
66. A plant produced by the method of any of embodiments 1-65.
67. A progeny of the plant of embodiment 66.
68. A seed of the plant of embodiment 66 or 67.

### VII. EXAMPLES

### Example 1: Modulation of Protein Activity Through an in-Frame Deletion

At least one desirable protein of interest in a plant is identified through methods known in the art, wherein the activity of the at least one protein of interest is responsible for a desirable agronomic trait. Identifying proteins of interest is accomplished through functional gene studies and analysis of available genetic databases. For example, a protein of interest can be identified by determining if there is gene with a known loss of function mutation that alters a desirable agronomic trait.

After identification of a protein of interest, one or more gRNAs are designed to target one or more exons in the gene sequence encoding the protein of interest. A gene editing system comprising a Cas nuclease and the one or more guide RNAs is delivered to a plant or plant part in order to edit the one or more exons targeted by the gRNAs. Editing by the gene editing system results in a deletion of one or more amino acids in the protein of interest (i.e., an in-frame deletion). Validation of the gene editing is conducted according to methods known in the art, such as gene sequencing. The edited plant or plant part comprising an in-frame deletion is regenerated, grown, and/or propagated and assessed for a phenotype associated with the desirable agronomic trait.

### Example 2: Generation of an Allelic Series Through in-Frame Deletions

After identifying a protein of interest, as described in Example 1, multiple gRNAs are designed to target one or more exons in the gene sequence encoding the protein of interest. A gene editing system comprising a Cas nuclease and the multiple gRNAs are delivered to a plurality of plants or plant parts in order to generate a series of edits in the one or more exons targeted by the gRNAs. Editing by the gene editing system results in a series of different amino acid deletions in the protein of interest (i.e., in-frame deletions) across the plurality of plants or plant parts. Validation of the gene editing is conducted according to methods known in the art, such as gene sequencing. The plurality of edited plants or plant parts, each comprising a different in-frame deletion, are regenerated, grown, and/or propagated and assessed for a phenotype associated with the desirable agronomic trait. A range of phenotypes for the desirable agronomic trait are observed across the plurality of plants.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of modulating the activity of a protein of interest in a plant or plant part comprising providing a Cas nuclease and a guide RNA for the Cas nuclease to the plant or plant part,
wherein the guide RNA is complementary to a target sequence in a target site,
wherein the target site comprises one or more codons of a plant gene encoding a protein of interest,
wherein editing by the Cas nuclease and the guide RNA results in deletion of one or more amino acids in the protein of interest.

2. A method of creating an allelic series of a plant gene comprising providing a Cas nuclease and a guide RNA for the Cas nuclease to each of a plurality of plants,
wherein the guide RNA is complementary to a target sequence in a target site,
wherein the target site comprises one or more codons of a plant gene encoding a protein of interest,
wherein editing by the Cas nuclease and the guide RNA generates a series of different amino acid deletions in the protein of interest across the plurality of plants.

3. The method of claim 2, further comprising selecting progeny of the plurality of plants,
wherein each of the progeny contain different amino acid deletions in the protein of interest.

4. The method of claim 3, wherein the selected progeny exhibit a range of phenotypes.

5. The method of claim 4, wherein the range of phenotypes is a spectrum of intermediate phenotypes for a quantitative agronomic trait.

6. The method of claim 4 or claim 5, wherein one or more of the selected progeny exhibits an improvement in an agronomic trait.

7. The method of claim 1, further comprising selecting a progeny from the plant,
wherein the progeny exhibits an improvement in an agronomic trait due to the deletion of one or more amino acids in the protein of interest.

8. The method of any of claims 3-7, further comprising backcrossing the selected progeny to remove the Cas nuclease and the guide RNA.

9. The method of any of claims 3-8, wherein the selected progeny no longer contains the Cas nuclease and the guide RNA.

10. The method of any one of claims 2-9, wherein the method comprises providing two or more, three or more, four or more, five or more, six or more, seven or more, or eight or more guide RNAs to the plurality of plants.

11. The method of claim 10, wherein each of the guide RNAs targets one or more target sites within a single exon of the plant gene encoding the protein of interest.

12. The method of claim 10, wherein each of the guide RNAs targets one or more target sites in different exons of the plant gene encoding the protein of interest.

13. The method of any of claims 1-12, wherein the guide RNA targets an evolutionary conserved sequence in the protein of interest.

14. The method of any of claims 1-13, wherein the deletion of one or more amino acids in the protein of interest comprises an in-frame deletion.

15. The method of claim 14, wherein the in-frame deletion comprises a deletion of a multiple of 3 nucleotides that results in the deletion of one or more codons of the plant gene encoding the protein of interest.

16. The method of claim 15, wherein the in-frame deletion further comprises a partial deletion of one codon and a synonymous mutation in an adjacent codon.

17. The method of claim 14, claim 15, or claim 16, wherein the in-frame deletion creates an allelic variant of the plant gene encoding the protein of interest.

18. The method of any of claims 14-17, wherein the in-frame deletion comprises a deletion of two, three, four, five, six, seven, eight, nine, or ten codons from the plant gene encoding the protein of interest.

19. The method of any of claims 14-18, wherein the in-frame deletion comprises a deletion of one or more codons encoding cysteine from the plant gene encoding the protein of interest.

20. The method of any of claims 14-19, wherein the in-frame deletion comprises a deletion of a stop codon from the plant gene encoding the protein of interest.

21. The method of claim 20, wherein deletion of the stop codon results in an increase in the length of the protein of interest.

22. The method of any of claims 14-21, wherein the in-frame deletion comprises a deletion of three, six, nine, 12, 15, 18, 21, 24, 27, or 30 nucleotides comprising one or more codons from the plant gene encoding the protein of interest.

23. The method of any of claims 1-22, wherein editing by the Cas nuclease and the guide RNA increases or reduces the biological activity of the protein of interest.

24. The method of any of claims 1-23, wherein editing by the Cas nuclease and the guide RNA results in a different structural isoform of the protein of interest.

25. The method of any of claims 1-24, wherein editing by the Cas nuclease and the guide RNA results in truncation of the protein of interest.

26. The method of any of claims 1-25, wherein editing by the Cas nuclease and the guide RNA results in a N-terminal or C-terminal truncation of the protein of interest.

27. The method of any of claims 1-26, wherein editing by the Cas nuclease and the guide RNA removes a site of a post-translational modification or co-translational modification from the protein of interest.

28. The method of any of claims 1-27, wherein editing by the Cas nuclease and the guide RNA results in decreased post-translational modification or co-translational modification of the protein of interest.

29. The method of any of claims 1-28, wherein editing by the Cas nuclease and the guide RNA increases turnover of the protein of interest.

30. The method of any of claims 1-28, wherein editing by the Cas nuclease and the guide RNA decreases turnover of the protein of interest.

31. The method of any of claims 1-30, wherein editing by the Cas nuclease and the guide RNA stabilizes an mRNA transcript encoding the protein of interest.

32. The method of any of claims 1-30, wherein editing by the Cas nuclease and the guide RNA destabilizes an mRNA transcript encoding the protein of interest.

33. The method of any of claims 1-32, wherein editing by the Cas nuclease and the guide RNA results in alternative splicing of an mRNA transcript encoding the protein of interest.

34. The method of any of claims 1-33, wherein editing by the Cas nuclease and the guide RNA removes a binding site on the protein of interest.

35. The method of claim 34, wherein the binding site removed from the protein of interest is for a protein, nucleic acid, lipid, carbohydrate or small molecule.

36. The method of any of claims 1-35, wherein editing by the Cas nuclease and the guide RNA alters the subcellular localization of the protein of interest.

37. The method of any of claims 1-36, wherein editing by the Cas nuclease and the guide RNA alters the intercellular transport of the protein of interest.

38. The method of any of claims 1-37, wherein editing by the Cas nuclease and the guide RNA decreases the level of the protein of interest.

39. The method of claim 38, wherein the level of the protein of interest is decreased by at least 1%, at least 5%, at least 10%, at least 15%, at least 20%, at least 30%, at least 40%, at least 50%, at least 70%, at least 80%, or at least 90%.

40. The method of any of claims 1-39, wherein editing by the Cas nuclease and the guide RNA increases the level of the protein of interest.

41. The method of claim 40, wherein the level of the protein of interest is increased by at least 1%, at least 5%, at least 10%, at least 15%, at least 20%, at least 30%, at least 40%, at least 50%, at least 70%, at least 80%, at least 90%, at least 100%, at least 200%, at least 300%, at least 400%, at least 500%, at least 600%, at least 700%, at least 800%, or up to 900%.

42. The method of any of claims 1-41, wherein the Cas nuclease is a Cas9, Cas12a (Cpf1), Cas12c, Cas12d, Cas12e, Cas12b, Cas12h, Cas12i, Cas12j, Cas12f, or an engineered Cas nuclease.

43. The method of any of claims 1-42, wherein the Cas nuclease is a Cas12i nuclease.

44. The method of claim 43, wherein the Cas12i nuclease is derived from Lachnospiraceae.

45. The method of any of claims 1-42, wherein the Cas nuclease is a Cas12a (Cpf1) nuclease.

46. The method of claim 43, wherein the Cas12a nuclease is derived from Francisella novicida, Acidaminococcus sp, or Lachnospiraceae.

47. The method of any of claims 1-46, wherein expression of the Cas nuclease is driven by a constitutive, inducible, tissue-specific or developmental promoter.

48. The method of any of claims 1-47, wherein the Cas nuclease is codon-optimized for expression in dicots or monocots.

49. The method of any of claims 1-48, wherein the Cas nuclease and the guide RNA are provided by particle bombardment of a plant, plant part or plurality of plants.

50. The method of any of claims 1-49, wherein the Cas nuclease and the guide RNA are provided by Agrobacterium-mediated transformation of the plant, plant part or plurality of plants.

51. The method of any of claims 1-50, wherein the guide RNA is provided by application of a composition comprising the guide RNA to the plant, plant part or plurality of plants.

52. The method of any of the claims 1-51, further comprising providing a donor template.

53. The method of claim 52, wherein insertion of the donor template results in the precise deletion of one or more codons in the plant gene encoding the protein of interest.

54. The method of any of claims 1-53, wherein the Cas nuclease is associated with a reverse transcriptase.

55. The method of any of claims 5-54, wherein the agronomic trait is selected from the group consisting of herbicide tolerance, disease resistance, insect or pest resistance, increased grain yield, increased oil, altered plant maturity, enhanced stress tolerance, and altered morphological characteristics.

56. The method of any of claims 1-55, wherein the plant gene encoding the protein of interest is a developmental gene.

57. The method of any of claims 1-56, wherein the plant gene encoding the protein of interest exhibits dosage sensitivity.

58. The method of any of claims 1-57, wherein the plant gene encoding the protein of interest regulates the cell cycle, cell growth, stem cell fate or hormone signaling.

59. The method of any of claims 1-58, wherein the protein of interest is a signaling peptide or transcription factor.

60. The method of any of claims 1-59, wherein low or high levels of the protein of interest are associated with an agronomically undesirable phenotype.

61. The method of any of claims 1-60, wherein the protein of interest is involved in drought tolerance, disease resistance, pest resistance, stress tolerance, yield, shape, odor, texture, metabolite production, pigmentation, seed fecundity, endoreduplication, sugar content, pH, improved shelf life or storability, cell differentiation, branching, plant height, time to fruit set, or light reception.

62. The method of any of claims 1-61, wherein the plant or plurality of plants is selected from the group consisting of maize, rice, sorghum, rye, barley, wheat, pearl millet, foxtail millet, proso millet, fonio millet, teff, flax, oats, sugarcane, turfgrass, switchgrass, soybean, canola, alfalfa, sunflower, cotton, tobacco, tomato, peanut, potato, cannabis, a forage crop, an industrial crop, a woody crop, a biomass crop, and Arabidopsis.

63. The method of any of claims 1-62, wherein the plant part is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

64. The method of any of claims 1-63, wherein the plant, plant part or plurality of plants is a crop plant.

65. The method of any of claims 1-64, wherein the plant, plant part or plurality of plants is a monocot or a dicot.

66. A plant produced by the method of any of claims 1-65.

67. A progeny of the plant of claim 66.

68. A seed of the plant of claim 66 or 67.
